(19) United States
(12) Patent Application Publication
(10) Pub. No.: US 2016/0067071 A1
(43) Pub. Date: Mar. 10, 2016

(54) BIORESORBABLE BIOPOLYMER STENT

(71) Applicant: Trustees of Tufts College, Medford, MA (US)

(72) Inventors: Rodrigo Jose, Medford, MA (US); David L. Kaplan, Concord, MA (US); Mark Iafrati, Wellesley, MA (US); Eun Seok Gil, Lexington, MA (US); Gary G. Leisk, Wilmington, MA (US)

(21) Appl. No.: 14/785,104

(22) PCT Filed: Apr. 24, 2014
(86) PCT No.: PCT/US14/35348

§ 371 Date: Oct. 16, 2015

Related U.S. Application Data
(60) Provisional application No. 61/815,519, filed on Apr. 24, 2013.

(51) Int. Cl. A61F 2/93; A61F 2/844

(52) U.S. Cl. CPC A61F 2/93; A61F 2/844; A61F 2002/825

(57) ABSTRACT

A bioresorbable biopolymer stents can be deployed within a blood vessel and resorbed by the body over a predetermined time period after the blood vessel has been remodeled. A ratcheting biopolymer stent can include a ratcheting mechanism that allows the biopolymer stent to be deployed on a small diameter configuration and then expanded to a predefined larger diameter configuration wherein after expansion, the ratcheting mechanism locks the biopolymer stent in the expanded configuration. A folding biopolymer stent can be deployed in a folded, small diameter configuration and then expanded to an unfolded configuration having a larger diameter. The bioresorbable biopolymer can include silk fibroin and blend that include silk fibroin materials.

### CROSS-REFERENCE TO RELATED APPLICATIONS

This application claims priority to U.S. provisional patent application Ser. No. 61/815,519, filed on Apr. 24, 2013, the disclosure of which is hereby incorporated in its entirety.

### STATEMENT REGARDING FEDERALLY SPONSORED RESEARCH

This invention was made with government support under grant EB002520 awarded by the National Institutes of Health. The government has certain rights in the invention.

### BACKGROUND

1. Technical Field of the Invention

The invention is directed to bioresorbable biopolymer stents and methods and systems for deploying these stents. Specifically, the invention is directed to ratcheting and unfolding bioresorbable biopolymer stents that provide for increased diameter. The bioresorbable biopolymer can include silk fibroin and blend that include silk fibroin materials.

2. Description of the Prior Art

Stents provide an immediate mechanical opening which improves vessel patency and prevents restenosis after implantation. However, the goals of stenting are achieved within weeks to months after implantation (see Waksman R, Biodegradable Stents: They Do their Job and Disappear: Why Bioabsorbable Stents?, J Invasive Cardiol., 2006, 18(2): 70-74). Recent research suggests that the response of the vessel wall to stent deployment reveals the role of the implant can be temporary because the mechanical stresses produced by stent implantation induces remodeling of the vessel walls (see Freeman et al., A link between stent radial forces and vascular wall remodeling: the discovery of an optimal stent radial force for minimal vessel restenosis, Connective Tissue Research, 2010, 51(4): 314-326). The continued presence of the stent becomes unnecessary and in some cases becomes deleterious. Current stent technology permanently remains in the vessel, which introduces many limitations including the risk of early and late thrombosis requiring the permanent use of P2Y₁₂ inhibitors for antiplatelet drug treatment (see Van Belle et al, Drug-eluting stents: trading restenosis for thrombosis?, J Thrombosis and Haemostasis, 2007, Suppl 1(January):238-245). Furthermore, current permanent stents generate additional concerns about late malapposition, hypersensitivity reactions, incomplete endothelialization or long-term impairment of endothelial response, elimination of vasomotion within the stented segment, and target lesion revascularization rates (see Gomes et al., Coronary stening and inflammation: implications for further surgical and medical treatment, Annals of Thoracic Surgery, 2006, 81(5): 1918-1925; see also Hofma et al., Increasing arterial wall injury after long-term implantation of two types of stent in a porcine coronary model, European Heart Journal, 1998, 19(4):601-609; Palmerini et al., Stent thrombosis with drug-eluting and bare-metal stents: evidence from a comprehensive network meta-analysis, Lancet, 2012, 379(9824):1393-1402).

A completely resorbable yet mechanically sufficient drug-eluting polymer stent that meets clinical applications of current metallic stents is not commercially available. The most clinically advanced biodegradable stents consist of magnesium alloys, with complete absorption in a few months (see Erbel et al., Temporary scaffolding of coronary arteries with bioabsorbable magnesium stents: a prospective, non-randomized multicenter trial, Lancet, 2007, 369(9576): 1869-1875). Medication coated stents reduce the risk of restenosis and blood clots, but current metal stents only facilitate limited release. Opening of affected arteries is performed via balloon angioplasty, stent placement, rotablation or cutting balloons. Balloon angioplasty is a procedure in which a small balloon at the tip of the catheter is inserted into an artery and is inflated to compress fatty plaque or blockage against the artery walls. This procedure is often complicated by vessel recoil and restenosis and requires a stent for support. Rotablation makes use of a high speed diamond-coated rotary catheter tip to aggressively grind plaque on the arterial walls, releasing plaque into the bloodstream. The cutting balloon catheter uses small blades score, slice and compress eroding tissue and plaque.

The first resorbable polymer stent implanted in humans, developed by Kyoto Medical Planning Company (Kyoto, Japan) was a balloon-mounted self-expanding design constructed from poly-L-lactic acid (PLLA), which degrades by bulk erosion (see Nishio et al., Long Term (>10 years) clinical outcomes of first-in-human biodegradable poly-1-lactic acid coronary stents, Circulation, 2012, 125(19):2343-2353). In the absorption process, hydrolysis of bonds between repeating lactide units produces lactic acid that enters the Krebs cycle and is metabolized to carbon dioxide and water. This device received a CE Mark in 2007 and is sold under the name REMEDY in Europe. The balloon-mounted deployment system requires expansion to be hastened by dilatation with contrast medium at a temperature of 80° C., which makes use cumbersome (see Nishio et al). Abbott Vascular (Santa Clara, Calif.) later developed the ABSORB polylactic acid everolimus-eluting stent producing clinical and imaging outcomes similar to those following metallic drug-eluting stents (see NIHR HSC, Bioresorbable stents for occlusive coronary artery disease, Birmingham:NIHR Horizon Scanning Centre (NIHR-HSC), Horizon Scanning Review, 2012). Although not available for sale in the United States, ABSORB received CE Mark in 2011. However, future development must target prevention of stent shrinkage exhibited by the ABSORB stent, after implantation in vivo (see Ormiston and Serruys, Bioabsorbable coronary stents, Circulation, 2009, 2(3):255-260).

Reva Medical Inc. (San Diego, Calif.) developed a resorbable stent using a tyrosine-derived polycarbonate polymer that metabolizes to amino acids, ethanol, and carbon dioxide (see Ormiston and Serruys). This is balloon expandable with a slide and lock (ratchet) design that allows stent expansion without material deformation. The REZORB first-in-man trial, which did not utilize a drug coating, had primary end points of major adverse events, such as, myocardial infarction, within 30 days (see Ormiston and Serruys). Further restenosis due to focal mechanical failures increased target lesion revascularization rate within 4 to 6 months (see Gonzalo and Macaya, Absorbable stent: focus on clinical applications and benefits, Vascular Health and Risk Management, 2012, 8:125-132). As a result, Reva is developing the ReZolve stent, a sirolimus-eluting revision with improved polymer strength.

The IDEAL stent, developed by Bioabsorbable Therapeutics Inc. (Menlo Park, Calif.) is a drug-eluting stent composed of poly(anhydride ester) salicylic acid (see Gonzalo and Macaya). The coating polymer is repeating salicylate molecules linked by adipic acid molecules while different linker molecules are used to join the stent backbone (see Ormiston and Serruys). This stent is designed to elute sirolimus but also releases salicylic acid as bonds are hydrolyzed during absorption. Absorption of the IDEAL stent, which is expected to be complete within 6 to 12 months, progresses by surface erosion (see Ormiston and Serruys). However, initial trials produced higher than expected intimal hyperplasia and restenosis necessitating design revisions (see Ormiston and Serruys). As a result, future revisions may include reducing strut thickness, percent wall coverage, and increasing sirolimus dosing.

Biotronik (Berlin, Germany) has made considerable advancements to balloon expandable magnesium alloy stents. These stents, which are laser cut from tubular magnesium WE-43 or AE21, generally exhibit better initial mechanical properties and radial strength compared to polymer variants (see Kwon D Y, Biodegradable stent, J Biomed Sci and Engineering, 2012, 05(04): 208-216). However, high rates of restenosis in the results of the PROGRESS AMS trial suggest loss of radial support during absorption happens prematurely (see Waksman et al., Early- and long-term intravascular ultrasound and angiographic findings after bioabsorbable magnesium stent implantation in human coronary arteries, JACC, 2009, 2(4): 312-320). Unlike polymer blends which undergo bulk erosion, absorption of magnesium stents occurs by surface erosion, which decreases strut thickness as the stent is absorbed (see Ormiston and Serruys). This may lead to an insufficient radial strength to counter the force of early remodeling (see Gonzalo and Macaya). Incomplete endothelialization which is characteristic of metallic degradable implants (see Ormiston and Serruys). Developments in this field are focused on perfecting control and tuning of degradation timing.

From the mechanical perspective, metallic stents which use deformation style deployment, rigidly maintain permanent diameters, and thus can potentially limit positive remodeling (see Ramcharitar and Serruys, Fully biodegradable coronary stents: progress to date, Amer J Cariovascular Drugs, 2008, 8(5): 305-314). Metallic stents which use self-expanding deployment fluctuate in diameter with vasomotion but do so by producing a shearing motion as struts slide past each other. The feature damages prior endothelialization and is a concern because damaged endothelial coverage is considered a main contributor to thrombosis following stent implantation (see Simons M, VEGF and restenosis: the rest of the story, Arteriosclerosis, thrombosis, and vascular boil., 29(4): 439-440).

### SUMMARY

The present invention is directed to resorbable stents that can replace permanent implants and can be used in cases where a physiological problem may be temporary. Since the role of a stent can be temporary once remodeling of the vessel walls has been completed, resorption of the implant would eliminate the associated risks of thrombosis. The use of resorbable stents made of degradable biomaterials which will progressively disappear after the vessel remodels, would remove the need for prolonged antiplatelet drugs once the stent has been resorbed. Resorbable stents allow the possibility of subsequent stent revascularization without the need to excise previous implants after tissue in-growth. In particular, this is useful in cases where patient vasculature will outgrow the diameter of permanent stent implants, such as when the patients are children or adolescents suffering from congenital disease or traumatic vascular injury.

There are advantages to using polymer-based resorbable stents over metal-based implants. Metal drug-eluting stents are limited to a thin polymer matrix for drug delivery. In contrast, a bioresorbable stent composed of biopolymers can deliver greater drug payloads over longer time periods thereby limiting the occurrence of localized restenosis throughout the process of implant resorption. Polymer stents can also allow segregation of different drugs throughout the bulk material for more complex drug release, to provide more control of stimulation of endothelialization and inhibition of smooth muscle. This approach also presents a unique opportunity to locally deliver multiple drugs over several time scales to treat a variety of clinical conditions. In addition to anti-restenotic drugs, polymer stents support various drug therapies such as more targeted chemotherapy.

Another benefit of polymer based stents is the lack of metallic artifacts to facilitate more precise diagnostic interpretation using magnetic resonance imaging or computed tomography. While modern metallic approaches offer initial strength and degradability, the degradation of alloys such as magnesium results in pronounced surface pitting which limits their use as implant material. Polymer based materials for use in resorbable stents benefit from their extensive use in tissue engineering. Unlike metallic stents, polymer materials have been developed as scaffolds with control, manipulation, and tailoring cellular processes and integration. Rather than the simple corrosion degradation of magnesium implants, polymer stents can facilitate true controllable tissue integration and implant resorption/degradation.

These and other capabilities of the invention, along with the invention itself, will be more fully understood after a review of the following figures, detailed description, and claims.

### BRIEF DESCRIPTION OF THE FIGURES

FIGS. 1A-1F show a ratcheting polymer stent according to a first embodiment of the invention.

FIGS. 2A-2E show a ratcheting polymer stent according to a second embodiment of the invention.

FIGS. 3A-3C show a ratcheting polymer stent according to a third embodiment of the invention.

FIGS. 4A-4C show a ratcheting polymer stent according to a fourth embodiment of the invention.

FIGS. 5A-5B show a folding polymer stent according to a fifth embodiment of the invention.

FIG. 6 shows a ratcheting polymer stent mounted on a catheter for insertion into a lumen according to some embodiments of the invention.

FIG. 7 shows a folding polymer stent mounted on a catheter for insertion into a lumen according to some embodiments of the invention.

FIG. 8A shows diagram of a ratcheting polymer stent mounted on a catheter for insertion into a lumen according to some embodiments of the invention.

FIGS. 8B and 8C show diagrammatic views of a folding polymer stent mounted on a catheter for insertion into a lumen according to some embodiments of the invention.

FIG. 9 shows a diagrammatic view of a method for mounting and deploying a folding polymer stent according to some embodiments of the invention.

FIGS. 10A-10C show a diagrammatic view of stented vessel dilation according to some embodiments of the invention.

FIGS. 11A and 11B show the compressive strength various embodiments of the present invention as compared with the prior art.

FIG. 12 shows a profile view of an exemplary stent loading tool compatible with various embodiments of the invention.

FIG. 13A shows a front view of the exemplary stent loading tool of FIG. 12.

FIG. 13B shows a close up front view of the pin holder and spacer pins engaged to hold a folding stent and placed within a sheath in accordance with various embodiments of the invention.

FIG. 14 shows an isometric view of an exemplary stent loading tool compatible with various embodiments of the invention.

FIG. 15 shows an isometric view of an exemplary stent loader compatible with various embodiments of the invention.

FIG. 16 shows a front isometric view of the exemplary stent loader depicted in FIG. 15.

FIG. 17 shows a direct front view of the exemplary stent loader depicted in FIG. 15.

FIG. 18 shows a side profile view of the exemplary stent loader depicted in FIG. 15.

### DETAILED DESCRIPTION OF PREFERRED EMBODIMENTS

The present invention is directed to bioresorbable biopolymer stents and methods and devices for deployment of the biopolymer stents. In accordance with some embodiments of the invention, all or portions of the stent can be formed from a biopolymer or biopolymer blend, for example, silk fibroin and blends and/or composite materials that include silk fibroin and a plasticizer. The stents can be fabricated according one of several embodiments that complement each other and provide for a broad range of stenting applications.

In accordance with some embodiments of the invention, the stent can include a ratcheting component that functions well in applications where it is desirable to provide a maximal increase in stent diameter, for example, when a surgeon must first traverse through vessels narrower than the target stent vessel.

In accordance with some embodiments of the invention, stent can be provided with a substantially solid wall construction that can be folded to provide a small diameter for insertion and unfolded to provide a larger diameter when set into place. The folding stent can be used in applications where it is desirable to minimize the risk of vessel erosion & restenosis. The stents function primarily to restore patency in vessels which have become occluded due to vascular disease or trauma. Both designs can be composed of the same or similar bulk materials.

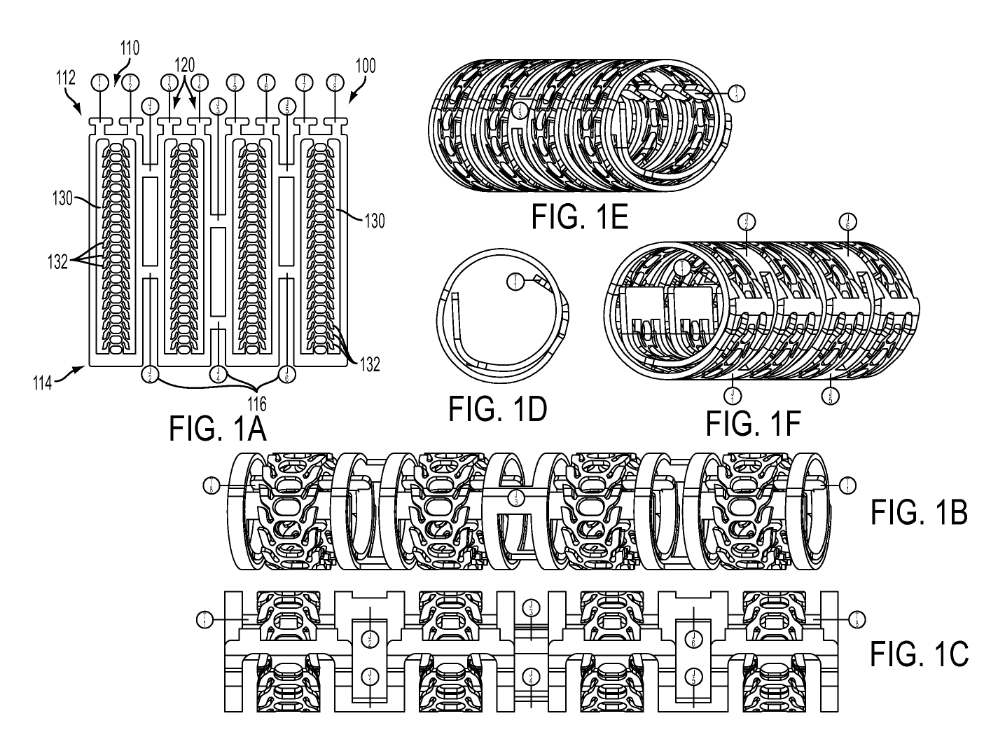

In accordance with some embodiments of the invention, the polymer stent can include one or more ratcheting mechanisms that allow the stent to be assembled into a small diameter configuration for insertion into the lumen of a vessel, such as a blood vessel. FIGS. 1A-1F, 2A-2E, 3A-3C and 4A-4C provide illustrative examples of polymer stents that include one or more ratcheting mechanisms according to some of the principles of the invention.

FIGS. 1A-1F show a ratcheting polymer stent 100 according to a first embodiment of the invention. The stent 100 can include one or more stent elements 110, for example, FIGS. 1A-1F show four stent elements 110, each being connected or joint to one or more adjacent stent elements 110 by a joint 116. The joints 116 can be cut to enable the axial length of the stent 100 to be reduced. Each stent element 110 can include a first end 112 and a second end 114. The first end 112 can include one or more tabs 120 that are adapted to fit into slots 130. The slots 130 can extend from the second end 114 toward the first end 112 and while the figure shows the slot 130 extending all the way to the first end 112, the slot can end before reaching the first end 112, for example, ending in the middle of the device. The slot 130 can also include one or more teeth 132 that interact with tab 120 to control the diameter of the stent 100. The teeth 132 can be configured with an angled surface that allows the tab 120 to more easily slide past the teeth 132 in one direction (e.g. to increase in diameter) but resist movement in the opposite direction (e.g. to resist compressive forces that would reduce its diameter). The teeth 132 can be resilient or flexible to enable the tab 120 to more easily move past in one direction than in the opposite direction. The teeth 132 can extend at an angle with respect to the length of the slot 130 such that each tooth 132 flexes in one direction as the tab 120 moves past it but engages the end of the tooth 132 that resist movement of the tab 120 in the opposite direction.

In the embodiment shown in FIGS. 1A-1F, each stent element 110 can be 5.0 mm wide by 30 mm long. When the stent 100 assembled into a tightly wound compressed tube, the stent 100 can have a minimum diameter of about 3.0 mm and can be expanded to a maximum diameter of about 10 mm. Each tab 120 can be “T” shaped and with the thinnest portion being approximately 1.5 mm wide. The thin portion of the tab 120 is adapted to fit in slot 130 which can be approximately 1.0 mm wide. The distance between each tab 120 and each slot 130 can be approximately 0.5 mm.

In these configurations, the tab 120 can be inserted into the slot 130 at a location close to the first end 112 with the second end curled around the inside of the stent 100 as shown in FIGS. 1B-1F. An outer sleeve can be used to hold the stent 100 in the compressed configuration for ease of insertion. After the stent 100 is inserted into the lumen of the vessel to be supported, the sleeve can be removed and an expansion force (e.g., such as that created by an expanding balloon) can be used to expand the stent 100 to the desired position. The number and location of the teeth 132 can be configured on regular intervals, for example, 100 micrometer increments, such that expanding the stent 100 by one tooth 132 increases the diameter by a predefined amount (in this example, 100/π micrometers).

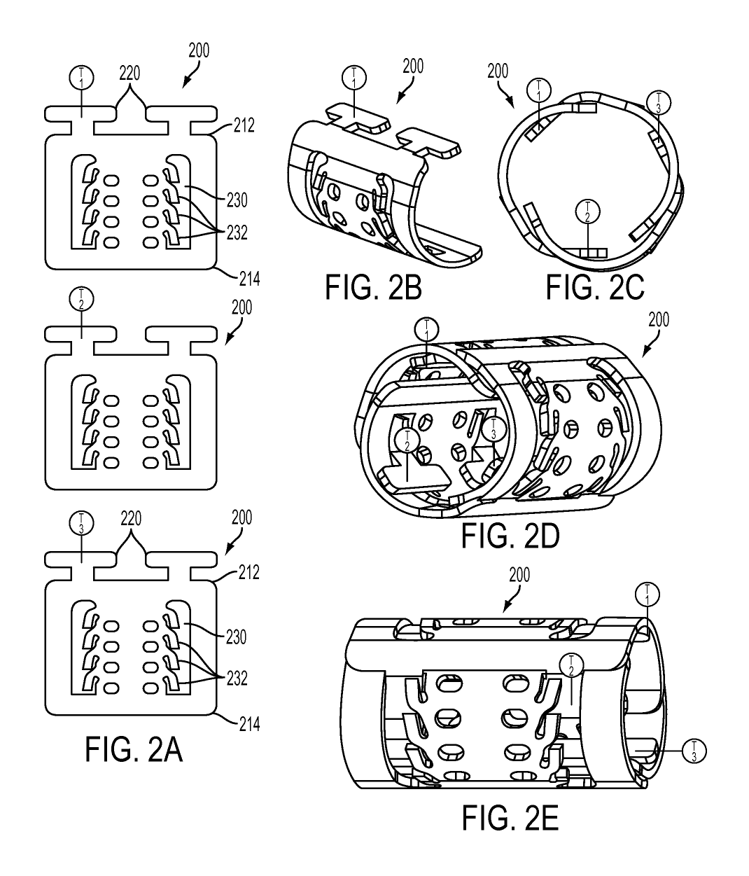

FIGS. 2A-2E show a ratcheting polymer stent 200 according to a second embodiment of the invention. The stent 200 can be formed by combining two or more stent elements 210 in an end to end configuration as shown in FIGS. 2B-2E. While these figures show three, relatively short stent elements 210 connected end to end for form a hollow tube more stent elements 210 can be used to produce a larger diameter stent 200 and longer stent elements 210 can be used to enable larger variations in expanded stent diameter. Stent elements 210 of differing lengths can be used together. In this embodiment, each stent element 210 can be similar in the stent elements 110 shown in FIGS. 1A-1F. Thus each stent element 210 can include a first end 212 and a second end 214, the first end 212 can include one or more tabs 220 that are adapted to fit into slots 230. The slots 230 extend from the second end 214 toward the first end 212 and while the figure shows the slot 230 extending all the way to the first end 212, the slot can end before reaching the first end 212, for example, ending in the middle of the stent element 210. In this embodiment, the tabs 220 from one element can be inserted into the slots 230 of an adjacent stent element 210 to form circular chain of stent elements 210 with the second end 214 on the inside. The slot 230 can also include one or more teeth 232 that interact with tab 220 to control the diameter of the stent 200. The teeth 232 can be configured with an angled surface that allows the tab 220 to more easily slide past the teeth 232 in one direction (e.g. to increase in diameter) but resist movement in the opposite direction (e.g. to resist compressive forces that would reduce its diameter). The teeth 232 can be resilient or flexible to enable the tab 220 to more easily move past in one direction than in the opposite direction. The teeth 232 can extend at an angle with respect to the length of the slot 230 such that each tooth 232 flexes in one direction as the tab 220 moves past it but engages the end of the tooth 232 that resist movement of the tab 220 in the opposite direction. While FIGS. 2A-2E show only 1 row of stent element 210 axially, longer stents 200 can be created by widening each stent element 210 or by joining two or more stent elements 210 as shown in FIGS. 1A-1F.

In the embodiment shown in FIGS. 2A-2E, each stent element 210 can have substantially the same dimensions as the stent elements 110 shown in FIGS. 1A-1F, however the length of stent element 210 can range from 4 mm to 10 mm

In these configurations, the tab 220 can be inserted into the slot 230 at a location close to the first end 212 with the second end curled around the inside of the stent 200 as shown in FIGS. 2B-2E. An outer sleeve can be used to hold the stent 200 in the compressed configuration for ease of insertion. After the stent 200 is inserted into the lumen of the vessel to be supported, the sleeve can be removed and an expansion force (e.g., such as that created by an expanding balloon) can be used to expand the stent 200 to the desired position. The number and/or location of the teeth 232 can be configured on regular intervals, for example, 100 micrometer increments, such that expanding the stent 100 by one tooth 132 increases the diameter by a predefined amount (in this example, 100/π micrometers).

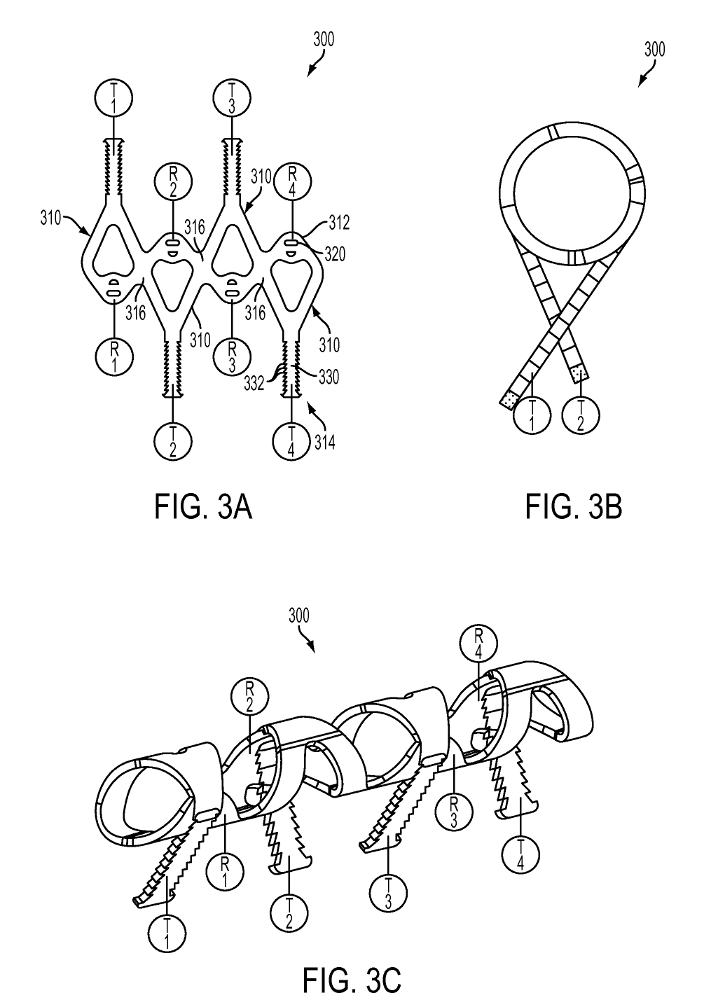

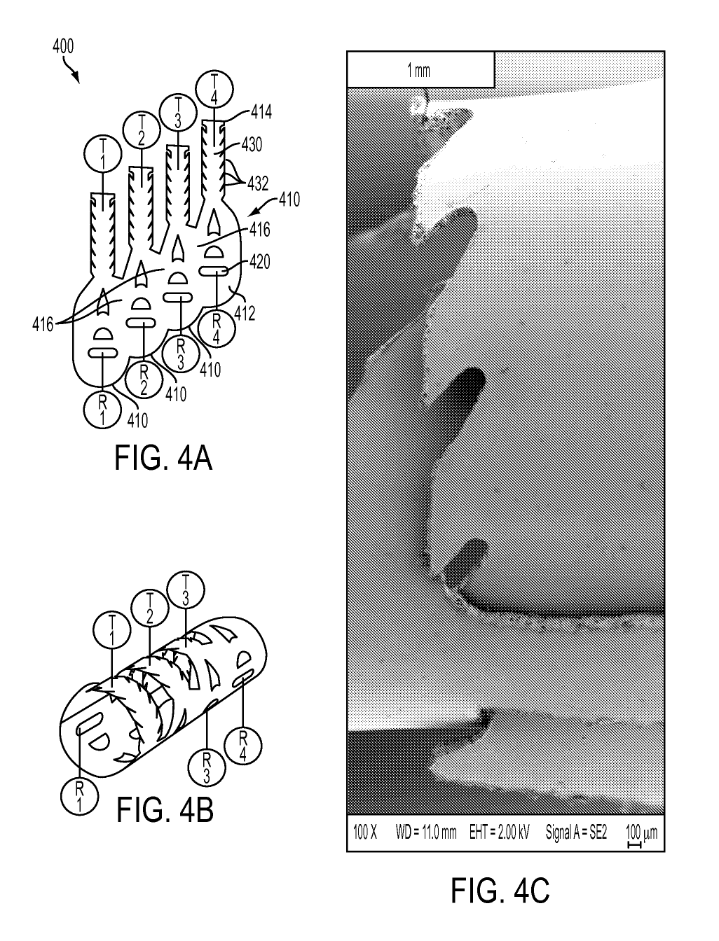

FIGS. 3A-3C show a ratcheting polymer stent 300 according to a third embodiment of the invention. The stent 300 can include one or more stent elements 310, for example, FIGS. 3A and 3C show four stent elements 310, each being connected or joint to one or more adjacent stent elements 310 by a common side or joint 316. The joints 316 can be cut to enable the axial length of the stent 300 to be reduced. Each stent element 310 can include a first end 312 and a second end 314, and the first end 112 can include one or more tongues or strips 330 that are adapted to fit into slots 320. The strips 330 extend from first end 312 to the second end 314. The strip 330 can also include one or more teeth 332 that interact with slot 320 to control the diameter of the stent 300. The teeth 332 can be configured with an angled surface that allows the slot 320 to more easily slide past the teeth 332 in one direction (e.g. to increase in diameter) but resist movement in the opposite direction (e.g. to resist compressive forces that would reduce its diameter). The teeth 332 can be resilient or flexible to enable the slot 320 to more easily move past each tooth 332 in one direction than in the opposite direction. The teeth 332 can extend at an angle with respect to the length of the strip 330 such that each tooth 332 flexes in one direction as the slot 320 moves past it but engages the end of the tooth 332 that resist movement of the slot 320 in the opposite direction. In this embodiment, the strips 330 of adjacent stent elements 310 can extend in opposite direction, however in other embodiments, such as shown in FIGS. 4A and 4B, the strips can extend in the same direction.

FIGS. 4A-4C show a ratcheting polymer stent 400 according to a fourth embodiment of the invention. The stent 400 can include one or more stent elements 410, for example, FIGS. 4A and 4C show four stent elements 410, each being connected or joint to one or more adjacent stent elements 410 by a common side or joint 416. The joints 416 can be cut to enable the axial length of the stent 400 to be reduced. Each stent element 410 can include a first end 412 and a second end 414, and the first end 412 can include one or more tongues or strips 430 that are adapted to fit into slots 420. The strips 430 extend from first end 412 to the second end 414. The strip 430 can also include one or more teeth 432 that interact with slot 420 to control the diameter of the stent 400. The teeth 432 can be configured with an angled surface that allows the slot 420 to more easily slide past the teeth 432 in one direction (e.g. to increase in diameter) but resist movement in the opposite direction (e.g. to resist compressive forces that would reduce its diameter). The teeth 432 can be resilient or flexible to enable the slot 420 to more easily move past each tooth 432 in one direction than in the opposite direction. The teeth 432 can extend at an angle with respect to the length of the strip 430 such that each tooth 432 flexes in one direction as the slot 420 moves past it but engages the end of the tooth 432 that resist movement of the slot 420 in the opposite direction. In this embodiment, the strips 430 of adjacent stent elements 410 can extend in the same direction, however in other embodiments, such as shown in FIGS. 3A and 3B, the strips 430 of adjacent stent elements 410 can extend in the same direction. In some embodiments of the invention, the stent elements 410 can be offset in a direction transverse to the cylindrical axis of the stent 400 (e.g., around the circumference of the tubular stent).

In accordance with some embodiments of the invention, the polymer stent design can employ micro ratchet slot and gear-rack mechanisms to enable large increases in diameter. Additionally, this design allows for deployment times within standard clinical limits and can be deployed faster than the 30-60 second requirement of current metal stents due to the lack of radial recoil associated with metal deformation. In some embodiments, provided stents may be deployed in less than 60 seconds, less than 50 seconds, less than 40 seconds, less than 30 seconds, less than 20 seconds, or less than 10 seconds. Fine tuning of the bulk material properties independently of the tab and slot geometry, which also enable the assembly, allows an additional level of control of the resorbable implant for extended periods of time. By utilizing the design to incorporate sophisticated yet simple mechanically articulating strut and joint assemblies, the initial flexibility and compliance of one piece material constructs can be tuned to control the bend flexibility while maintaining radial strength. The stent mechanical design can be optimized to meet desired functional requirements: (a) the stent can be deployed using standard clinical deployment tools, (b) the stent can be deployed with a low risk of injury by not requiring over-dilation or extended dilation times, (c) the stent can provide a predefined amount of radial strength because the radial strength is dependent on the assembled slot and tab which can be discretely measured at each position.

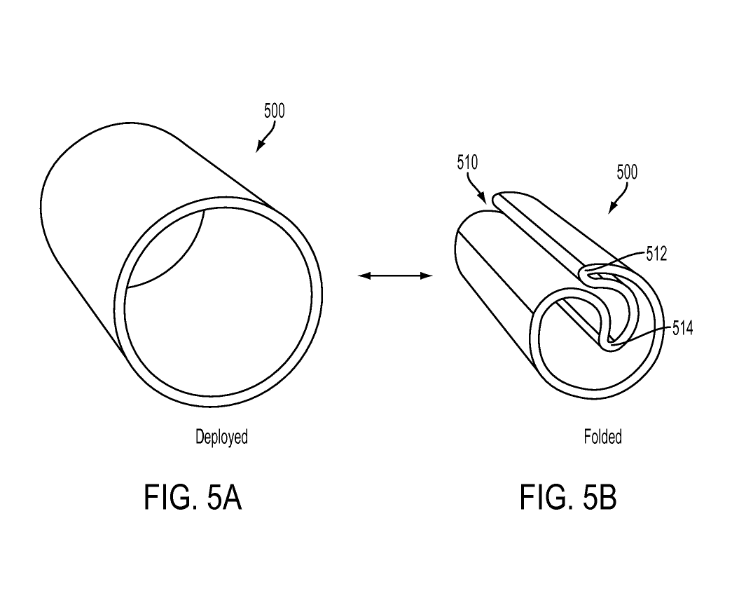

In accordance with some embodiments of the invention, the polymer stent can include one or more bends or folds that allow a cylindrical (or other shaped hollow tube) to be placed in a reduced diameter form for insertion into a lumen. After insertion, the folded polymer stent can be expanded to a larger diameter form. FIGS. 5A and 5B show an illustrative example of a folding polymer stent according to some principles of the invention.

FIGS. 5A and 5B show a folding polymer stent 500 according to some embodiments of the present invention. The folding polymer stent 500 can include a tubular bioresorbable polymer material having a predefined axial length. In accordance with some embodiments, the polymer tube 500 can range from 1 mm to 200 mm in length as desired (e.g., 1 mm to 150 mm, 1 mm to 100 mm, 5 mm to 100 mm, 10 mm to 100 mm, 20 mm to 100 mm, 30 mm to 90 mm, 40 mm to 80 mm, or 50 mm to 70 mm). The polymer tube 500 can include one or more bends or folds 512, 514 that enable the polymer tube 500 to be fitted into a smaller diameter lumen than when the polymer stent 500 is unfolded and fully expanded (e.g., compare FIG. 5A with FIG. 5B). While FIG. 5B shows an embodiment with 2 folds, additional folds can be provided to enable polymer tube 500 to be inserted into a smaller diameter lumen before expansion.

This embodiment of the invention can be used to provide a polymer stent 500 that is flexible yet radially robust. In accordance with some embodiments of the invention, the polymer stent can be tubular, having solid walls and initially permeable only to nutrients. As a result, the solid wall folding stent will not facilitate bulk tissue erosion driven restenosis. And while the increase in diameter after deployment can be less than the designs shown in FIGS. 1A-1F, 2A-2E, 3A-3C, and 4A-4C, the folding polymer stent can provide improved structural support where the risks of hyperplasia, restenosis, atherosclerosis, or vessel erosion are high.

The ratcheting stent designs (e.g., stents 100, 200, 300, 400) can be formed from a sheet of polymer material, such as a silk fibroin or blend of silk fibroin with other materials that can enhance the properties of the silk fibroin for specific applications. The sheet of polymer material can be cast (e.g., in PDMS molds) to produce a predefined thickness according to the application, for example, in the range of 100 micrometers to 500 micrometers, in the range of 150 micrometers to 450 micrometers, in the range from 200 micrometers to 400 micrometers, in the range from 250 micrometers to 350 micrometers. Where increased strength is desired, thicknesses above 300 micrometers can be used. Where faster resorbability is desired, thickness below 300 micrometers can be used. According to various embodiments, it may be desirable for thickness to be 300 micrometers or less. In accordance with some embodiments of the invention, the sheet material of a specific thickness (e.g., 150 to 300 micrometers) can die cut, micro-machined or laser cut into the desired configuration. In accordance with other embodiments of the invention, the polymer material can be cast directly into the desired configuration. The polymer stents in flat form can be distributed to healthcare providers for assembly and insertion as needed. After the polymer stent is formed, the flexible material can be assembled by hand or machine into small diameter tubes, using the ratcheting mechanism (and in some embodiments, assisted by an outer sheath) to hold the stent in its small diameter form prior to insertion. After insertion, any outer sheath can be removed and the polymer stent can be expanded in place to provide the desired internal diameter for fluid flow and structural support. The axial length of the stent can be selected according to the application and the needs of the patient to be treated. In accordance with some embodiments of the invention, the axial length of the ratcheting stent can be in the range from 1 millimeter to 50 millimeters, in the range from 10 mm to 40 mm, in the range from 20 mm to 30 mm. In accordance with some embodiments of the invention, the ratcheting stent can be formed from a long sheet that provides 2 or more stent segments that can be separated to provide a polymer stent of a predefined length.

The folding stent designs (e.g., stent 500) can be formed by casting on a round form, for example, by dipping the round form into the bulk polymer solution. The form can dipped more than one time to build up multiple layers of a tubular film on the form. The form can be tapered or coated with a non-stick coating that allows the tubular stent to be removed from the form. In accordance with some embodiments, the form can include a Teflon coated stainless steel rod ranging from 650 micrometers to 4,500 micrometers in diameter. The polymer stents can be produced in a series of discrete diameters, for example, as part of a kit or collection that can be used as need in an operating room setting, while ensuring proper vessel fit and reducing the risk of over-dilation deployment injury. The tubular layers of polymer material can be cast in a predefined thickness according to the application, for example, in the range of 100 micrometers to 500 micrometers, in the range of 150 micrometers to 450 micrometers, in the range from 200 micrometers to 400 micrometers, in the range from 250 micrometers to 350 micrometers. Where increased strength is desired, thicknesses above 300 micrometers can be used. Where faster resorbability is desired, thickness below 300 micrometers can be used. The axial length of the stent can be selected according to the application and the needs of the patient to be treated. In accordance with some embodiments of the invention, the axial length of the folding stent can be in the range from 1 millimeter to 50 millimeters, in the range of 5 mm to 45 mm, in the range from 10 mm to 40 mm, in the range of 15 mm to 35 mm, in the range from 20 mm to 30 mm. In accordance with some embodiments of the invention, the folding stent can be formed from a long tube that provides 2 or more stent segments that can be separated to provide a polymer stent of a predefined length. In accordance with some embodiments of the invention, the folding stent can be formed from a long, solid walled tube that is cut to a predefined length.

### Materials

In accordance with some embodiments of the invention, the bulk material, i.e., polymeric material, of the stent comprises silk fibroin. As used herein, the term “silk fibroin” or “fibroin” includes silkworm fibroin and insect or spider silk protein. See e.g., Lucas et al., 13 Adv. Protein Chem. 107 (1958). Any type of silk fibroin can be used according to aspects of the present invention. Silk fibroin produced by silkworms, such as Bombyx mori, is the most common and represents an earth-friendly, renewable resource. For instance, silk fibroin used in can be attained by extracting sericin from the cocoons of B. mori. Organic silkworm cocoons are also commercially available. There are many different silks, however, including spider silk (e.g., obtained from Nephila clavipes), transgenic silks, genetically engineered silks (recombinant silk), such as silks from bacteria, yeast, mammalian cells, transgenic animals, or transgenic plants, and variants thereof, that can be used. See for example, WO 97/08315 and U.S. Pat. No. 5,245,012, content of both of which is incorporated herein by reference in its entirety. In some embodiments, silk fibroin can be derived from other sources such as spiders, other silkworms, bees, and bioengineered variants thereof. In some embodiments, silk fibroin can be extracted from a gland of silkworm or transgenic silkworms. See for example, WO2007/098951, content of which is incorporated herein by reference in its entirety. In some embodiments, silk fibroin is free, or essentially free, of sericin.

The silk fibroin solution can be prepared by any conventional method known to one skilled in the art. For example, B. mori cocoons are boiled for about 30 minutes in an aqueous solution. In one embodiment, the aqueous solution is about 0.02M Na₂CO₃. The cocoons are rinsed, for example, with water to extract the sericin proteins and the extracted silk fibroin is dissolved in an aqueous salt solution. Salts useful for this purpose include lithium bromide, lithium thiocyanate, calcium nitrate or other chemicals capable of solubilizing silk fibroin. In some embodiments, the extracted silk fibroin is dissolved in about 8M -12 M LiBr solution. The salt is consequently removed using, for example, dialysis. According to various embodiments, the boil time of B. Mori cocoons may be varied in order to adjust the molecular weight of the silk fibroin material, for example, to alter the resorption characteristics and drug release profile of provided stents.

If necessary, the solution can then be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, M W CO 3500) can be used. However, any dialysis system can be used. The dialysis can be performed for a time period sufficient to result in a final concentration of aqueous silk fibroin solution between about 10% to about 30%. In most cases dialysis for 2-12 hours can be sufficient. See, for example, International Patent Application Publication No. WO 2005/012606, the content of which is incorporated herein by reference in its entirety.

Alternatively, the silk fibroin solution can be produced using organic solvents. Such methods have been described, for example, in Li, M., et al., J. Appl. Poly Sci. 2001, 79, 2192-2199; Min, S., et al. Sen'I Gakkaishi 1997, 54, 85-92; Nazarov, R. et al., Biomacromolecules 2004 May-June; 5(3):718-26, content of all which is incorporated herein by reference in their entirety. An exemplary organic solvent that can be used to produce a silk fibroin solution includes, but is not limited to, hexafluoroisopropanol (HFIP). See, for example, International Application No. WO2004/000915, content of which is incorporated herein by reference in its entirety. Accordingly, in some embodiments, the solution comprising the silk fibroin comprises an organic solvent, e.g., HFIP. In some other embodiments, the solution comprising the silk fibroin is free or essentially free of organic solvents.

Generally, any amount of silk fibroin can be present in the solution. For example, amount of silk fibroin in the solution can be from about 1% (w/v) to about 50% (w/v) of silk fibroin, e.g., silk fibroin. In some embodiments, the amount of silk fibroin in the solution can be from about 1% (w/v) to about 35% (w/v), from about 1% (w/v) to about 30% (w/v), from about 1% (w/v) to about 25% (w/v), from about 1% (w/v) to about 20% (w/v), from about 1% (w/v) to about 15% (w/v), from about 1% (w/v) to about 10% (w/v), from about 5% (w/v) to about 25% (w/v), from about 5% (w/v) to about 20% (w/v), from about 5% (w/v) to about 15% (w/v). In some embodiments, the amount of silk fibroin in the solution is less than 5% (w/v). In some embodiments, the amount of silk fibroin in the solution is greater than 25% (w/v). Exact amount of silk fibroin in the silk fibroin solution can be determined by drying a known amount of the silk fibroin solution and measuring the mass of the residue to calculate the solution concentration.

In some embodiments, the bulk material of the stent comprises one or more (e.g., one, two, three, four, five or more) additives. Without wishing to be bound by a theory, additives can provide one or more desirable properties to the stent, e.g., strength, flexibility, ease of processing and handling, biocompatibility, bioresorability, lack of air bubbles, surface morphology, and the like. The additive can be covalently or non-covalently linked with silk fibroin and can be integrated homogenously or heterogeneously within the bulk material. The additive can be in any physical form. For example, the additive can be in the form of a particle (e.g., microparticle or nanoparticle), a fiber, a film, a gel, a mesh, a mat, a non-woven mat, a powder, a liquid, or any combinations thereof. The bulk material of the stent containing the additive can be formulated by mixing one or more additives with the silk fibroin-fibroin solution used to make the stent.

Without limitations, the additive can be selected from the group consisting of anti-proliferative agents, biopolymers, nanoparticles (e.g., gold nanoparticles), proteins, peptides, nucleic acids (e.g., DNA, RNA, siRNA, modRNA), nucleic acid analogs, nucleotides, oligonucleotides, peptide nucleic acids (PNA), aptamers, antibodies or fragments or portions thereof (e.g., paratopes or complementarity-determining regions), antigens or epitopes, hormones, hormone antagonists, growth factors or recombinant growth factors and fragments and variants thereof, cell attachment mediators (such as RGD), cytokines, enzymes, small molecules, antibiotics or antimicrobial compounds, toxins, therapeutic agents and prodrugs, small molecules and any combinations thereof.

Ratio of the silk fibroin to the total amount of additives in the bulk material can range from 100:1 to 1:100. For example, the ratio of silk fibroin to additive can range from 50:1 to 1:50, from 25:1 to 1:25, from 20:1 to 1:20, from 15:1 to 1:15, from 10:1 to 1:10, or from 5:1 to 1:5. In some embodiments, the ratio of silk fibroin to additive can be from 5:1 to 1:1. In one embodiment, the ratio of silk fibroin to additive can be 3:1. The ratio can be molar ratio, weight ratio, or volume ratio.

In some embodiments, the additive is a plasticizer. As used herein, the term “plasticizer” is intended to designate a compound or a mixture of compounds that can increase flexibility, processability and extensibility of the polymer in which it is incorporated. A plasticizer can reduce the viscosity of the melt, lower the second order transition temperatures and the elastic modulus of the product. Suitable plasticizers include, but are not limited to, low molecular weight polyols having aliphatic hydroxyls such as ethylene glycol; propylene glycol; propanetriol (i.e., glycerol); glyceryl monostearate; 1,2-butylene glycol; 2,3-butylene glycol; styrene glycol; polyethylene glycols such as diethylene glycol, triethylene glycol, tetraethylene glycol and other polyethylene glycols having a molecular weight of about 1,000 or less; polypropylene glycols of molecular weight 200 or less; glycol ethers such as monopropylene glycol monoisopropyl ether; propylene glycol monoethyl ether; ethylene glycol monoethyl ether; diethylene glycol monoethyl ether; ester-type plasticizers such as sorbitol lactate, ethyl lactate, butyl lactate, ethyl glycolate, allyl glycolate; and amines such as monoethanolamine, diethanolamine, triethanolamine, monisopropanolamine, triethylenetetramine, 2-amino-2-methyl-1,3-propanediol, polymers and the like. In one embodiment, the plasticizer can include glycerol.

In some embodiments, the additive is a polymer. In some embodiments, the polymer is a biocompatible polymer. As used herein, the term “biocompatible polymer” refers to any polymeric material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood. Exemplary biocompatible polymers include, but are not limited to, a poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, fibronectin, keratin, polyaspartic acid, alginate, chitosan, chitin, hyaluronic acid, pectin, polyhydroxyalkanoates, dextrans, and polyanhydrides, polyethylene oxide (PEO), poly(ethylene glycol) (PEG), triblock copolymers, polylysine, alginate, polyaspartic acid, any derivatives thereof and any combinations thereof. Other exemplary biocompatible polymers amenable to use according to the present disclosure include those described for example in U.S. Pat. No. 6,302,848; U.S. Pat. No. 6,395,734; U.S. Pat. No. 6,127,143; U.S. Pat. No. 5,263,992; U.S. Pat. No. 6,379,690; U.S. Pat. No. 5,015,476; U.S. Pat. No. 4,806,355; U.S. Pat. No. 6,372,244; U.S. Pat. No. 6,310,188; U.S. Pat. No. 5,093,489; U.S. Pat. No. 387,413; U.S. Pat. No. 6,325,810; U.S. Pat. No. 6,337,198; U.S. Pat. No. 6,267,776; U.S. Pat. No. 5,576,881; U.S. Pat. No. 6,245,537; U.S. Pat. No. 5,902,800; and U.S. Pat. No. 5,270,419, content of all of which is incorporated herein by reference.

In some embodiments, the biocompatible polymer is PEG or PEO. As used herein, the term “polyethylene glycol” or “PEG” means an ethylene glycol polymer that contains about 20 to about 2000000 linked monomers, typically about 50-1000 linked monomers, usually about 100-300. PEG is also known as polyethylene oxide (PEO) or polyoxyethylene (POE), depending on its molecular weight. Generally PEG, PEO, and POE are chemically synonymous, but historically PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, the term PEG is intended to be inclusive and not exclusive. The term PEG includes poly(ethylene glycol) in any of its forms, including alkoxy PEG, difunctional PEG, multiarmed PEG, forked PEG, branched PEG, pendent PEG (i.e., PEG or related polymers having one or more functional groups pendent to the polymer backbone), or PEG With degradable linkages therein. Further, the PEG backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)m in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as biocompatible polymers.

Some exemplary PEGs include, but are not limited to, PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like. In some embodiments, PEG is of MW 10,000 Dalton. In some embodiments, PEG is of MW 100,000, i.e. PEO of MW 100,000.

In some embodiments, the polymer is a biodegradable polymer. As used herein, the term “biodegradable” describes a material which can decompose under physiological conditions into breakdown products. Such physiological conditions include, for example, hydrolysis (decomposition via hydrolytic cleavage), enzymatic catalysis (enzymatic degradation), and mechanical interactions. As used herein, the term “biodegradable” also encompasses the term “bioresorbable”, which describes a substance that decomposes under physiological conditions to break down to products that undergo bioresorption into the host-organism, namely, become metabolites of the biochemical systems of the host organism. As used herein, the terms “bioresorbable” and “bioresorption” encompass processes such as cell-mediated degradation, enzymatic degradation and/or hydrolytic degradation of the bioresorbable polymer, and/or elimination of the bioresorbable polymer from living tissue as will be appreciated by the person skilled in the art.

The term “biodegradable polymer”, as used herein, refers to a polymer that at least a portion thereof decomposes under physiological conditions. The polymer can thus be partially decomposed or fully decomposed under physiological conditions.

Exemplary biodegradable polymers include, but are not limited to, polyanhydrides, polyhydroxybutyric acid, polyorthoesters, polysiloxanes, polycaprolactone, poly(lactic-co-glycolic acid), poly(lactic acid), poly(glycolic acid), and copolymers prepared from the monomers of these polymers.

In some embodiments, the additive comprises a bioinert material. As used herein, the term “bioinert” refers to any material that once placed in vivo has minimal interaction with its surrounding tissue. Exemplary bioinert materials include, but are not limited to, gold, stainless steel, titanium, alumina, partially stabilized zirconia, and ultra-high molecular weight polyethylene.

In some embodiments, the additive can be selected from the group consisting of polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, and any combinations thereof.

In some embodiments, the additive can be a silk fibroin particle or powder. Various methods of producing silk fibroin particles (e.g., nanoparticles and microparticles) are known in the art. See for example, PCT Publication No. WO 2011/041395 and No. WO 2008/118133; U.S. App. Pub. No. U.S. 2010/0028451; U.S. Provisional Application Ser. No. 61/719,146, filed Oct. 26, 2012; and Wenk et al. J Control Release, Silk fibroin spheres as a platform for controlled drug delivery, 2008; 132: 26-34, content of all of which is incorporated herein by reference in their entirety.

In some embodiments, the additive is a silk fibroin fiber. In some embodiments, silk fibroin fibers could be chemically attached by redissolving part of the fiber in HFIP and attaching to stent. Use of silk fibroin fibers is described in, for example, US patent application publication no. US20110046686, content of which is incorporated herein by reference.

In some embodiments, the silk fibroin fibers are microfibers or nanofibers. In some embodiments, the additive is micron-sized silk fibroin fiber (10-600 μm). Micron-sized silk fibroin fibers can be obtained by hydrolyzing the degummed silk fibroin or by increasing the boing time of the degumming process. Alkali hydrolysis of silk fibroin to obtain micron-sized silk fibroin fibers is described for example in Mandal et al., PNAS, 2012, doi: 10.1073/pnas.1119474109; U.S. Provisional Application No. 61/621,209, filed Apr. 6, 2012; and PCT application no. PCT/US13/35389, filed Apr. 5, 2013, content of all of which is incorporated herein by reference. Because regenerated silk fibroin fibers made from HFIP silk fibroin solutions are mechanically strong. the regenerated silk fibroin fibers can also be used as additive.

In some embodiments, the silk fibroin fiber is an unprocessed silk fibroin fiber, e.g., raw silk fibroin or raw silk fibroin fiber. The term “raw silk fibroin” or “raw silk fibroin fiber” refers to silk fibroin fiber that has not been treated to remove sericin, and thus encompasses, for example, silk fibroin fibers taken directly from a cocoon Thus, by unprocessed silk fibroin fiber is meant silk fibroin, obtained directly from the silk fibroin gland. When silk fibroin, obtained directly from the silk fibroin gland, is allowed to dry, the structure is referred to as silk fibroin I in the solid state. Thus, an unprocessed silk fibroin fiber comprises silk fibroin mostly in the silk fibroin I conformation. A regenerated or processed silk fibroin fiber on the other hand comprises silk fibroin having a substantial silk fibroin II or beta-sheet crystallinity.

In some embodiment, the bulk material of the stent can comprise a biologically active agent. As used herein, the term “biological activity” or “bioactivity” refers to the ability of a molecule or composition to affect a biological sample. Biological activity can include, without limitation, elicitation of a stimulatory, inhibitory, regulatory, toxic or lethal response in a biological assay. For example, a biological activity can refer to the ability of a compound to modulate the effect/activity of an enzyme, block a receptor, stimulate a receptor, modulate the expression level of one or more genes, modulate cell proliferation, modulate cell division, modulate cell morphology, or any combination thereof. In some instances, a biological activity can refer to the ability of a compound to produce a toxic effect in a biological sample. The bulk material of the stent containing the active agent can be formulated by mixing one or more active agents with the silk fibroin-fibroin solution used to make the stent.

Without limitations, the active agent can be selected from small organic or inorganic molecules; saccharines; oligosaccharides; polysaccharides; biological macromolecules; peptides; proteins; peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. The active agent can be hydrophobic, hydrophilic, or amphiphilic.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is highly preferred that a small molecule have a molecular mass equal to or less than 700 Daltons.

Total amount of active agent in the bulk material can be from about 0.1 wt % to about 0.99 wt %, from about 0.1 wt % to about 70 wt %, from about 5 wt % to about 60 wt %, from about 10 wt % to about 50 wt %, from about 15 wt % to about 45 wt %, or from about 20 wt % to about 40 wt %, of the total weight of the bulk material.

Without wishing to be bound by a theory, the active agent can be covalently or non-covalently associated with the bulk material. In some embodiments, the active agent is distributed homogenously in the bulk material, e.g., silk fibroin matrix. In some embodiments, the active agent is absorbed/adsorbed on a surface of the stent.

Examples of biologically active compounds include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard, Cell Mol Life Sci,. 2003, 60(1):119-32 and Hersel U. et al., Biomaterials, 2003, 24(24):4385-415); YIGSR peptides; biologically active ligands; and substances that enhance or exclude particular varieties of cellular or tissue ingrowth.

In some embodiments, the active agent is a growth factor or cytokine A non-limiting list of growth factors and cytokines includes, but is not limited, to stem cell factor (SCF), granulocyte-colony stimulating factor (G-CSF), granulocyte-macrophage stimulating factor (GM-CSF), stromal cell-derived factor-1, steel factor, VEGF, TGFβ, platelet derived growth factor (PDGF), angiopoeitins (Ang), epidermal growth factor (EGF), bFGF, HNF, NGF, bone morphogenic protein (BMP), fibroblast growth factor (FGF), hepatocye growth factor, insulin-like growth factor (IGF-1), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factors (TNFα and TNFβ). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, P A: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In some embodiments, the active agent can be selected from anti-infectives such as antibiotics and antiviral agents; chemotherapeutic agents (i.e. anticancer agents); anti-rejection agents; anti-proliferative agents; analgesics and analgesic combinations; anti-inflammatory agents; erythropoietin (EPO); interferon α and γ; interleukins; tumor necrosis factor α and β; insulin, antibiotics; adenosine; cytokines; integrins; selectins; cadherins; insulin; hormones such as steroids; cytotoxins; prodrugs; immunogens; or lipoproteins.

In some embodiments, the active agent is a therapeutic agent. As used herein, the term “therapeutic agent” refers to a biological or chemical agent used for treating, curing, mitigating, or preventing deleterious conditions in a subject. The term “therapeutic agent” also includes substances and agents for combating a disease, condition, or disorder of a subject, and includes drugs, diagnostics, and instrumentation. “Therapeutic agent” also includes anything used in medical diagnosis, or in restoring, correcting, or modifying physiological functions. The terms “therapeutic agent” and “pharmaceutically active agent” are used interchangeably herein.

The therapeutic agent is selected according to the treatment objective and biological action desired. General classes of therapeutic agents include anti-microbial agents such as adrenergic agents, antibiotic agents or antibacterial agents, antiviral agents, anthelmintic agents, anti-inflammatory agents, antineoplastic agents, antioxidant agents, biological reaction inhibitors, botulinum toxin agents, chemotherapy agents, contrast imaging agents, diagnostic agents, gene therapy agents, hormonal agents, mucolytic agents, radioprotective agents, radioactive agents including brachytherapy materials, tissue growth inhibitors, tissue growth enhancers, and vasoactive agents. The therapeutic agent can be selected from any class suitable for the therapeutic objective. For example, if the objective is treating a disease or condition associated narrowing in a blood vessel or other tubular organ or structure, the therapeutic agent can include antithrombotic or fibrinolytic agents.

Exemplary pharmaceutically active compound include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

In some embodiments, the therapeutic agent can be selected from the group consisting of anti-infectives, chemotherapeutic agents, anti-rejection agents, analgesics and analgesic combinations, anti-inflammatory agents, hormones, growth factors, antibiotics, antiviral agents, steroids, bone morphogenic proteins, bone morphogenic-like proteins, epidermal growth factor, fibroblast growth factor, platelet derived growth factor (PDGF), insulin-like growth factor, transforming growth factors, vascular endothelial growth factor, and any combinations thereof.

In some embodiments, the therapeutic agent is an antithrombotic or fibrinolytic agent selected from the group consisting of anticoagulants, anticoagulant antagonists, antiplatelet agents, thrombolytic agents, thrombolytic agent antagonists, and any combinations thereof.

In some embodiments, the therapeutic agent is thrombogenic agent selected from the group consisting of thrombolytic agent antagonists, anticoagulant antagonists, pro-coagulant enzymes, pro-coagulant proteins, and any combinations thereof. Some exemplary thrombogenic agents include, but are not limited to, protamines, vitamin K1, amiocaproic acid (amicar), tranexamic acid (amstat), anagrelide, argatroban, cilstazol, daltroban, defibrotide, enoxaparin, fraxiparine, indobufen, lamoparan, ozagrel, picotamide, plafibride, tedelparin, ticlopidine, triflusal, collagen, and collagen-coated particles.

In some embodiments, the therapeutic agent is a vasodilator. A vasodilator can be selected from the group consisting of alpha-adrenoceptor antagonists (alpha-blockers), agiotensin converting enzyme (ACE) inhibitors, angiotensin receptor blockers (ARBs), beta2-adrenoceptor agonists (β2-agonists), calcium-channel blockers (CCBs), centrally acting sympatholytics, direct acting vasodilators, endothelin receptor antagonists, ganglionic blockers, nitrodilators, phosphodiesterase inhibitors, potassium-channel openers, renin inhibitors, and any combinations thereof. Exemplary vasodilator include, but are not limited to, prazosin, terazosin, doxazosin, trimazosin, phentolamine, phenoxybenzamine, benazepril, captopril, enalapril, fosinopril, lisinopril, moexipril, quinapril, ramipril, candesartan, eprosartan, irbesartan, losartan, olmesartan, telmisartan, valsartan, Epinephrine, Norepinephrine, Dopamine, Dobutamine, Isoproterenol, amlodipine, felodipine, isradipine, nicardipine, nifedipine, nimodipine, nitrendipine, clonidine, guanabenz, guanfacine, α-methyldopa, hydralazine, Bosentan, trimethaphan camsylate, isosorbide dinitrate, isosorbide mononitrate, nitroglycerin, erythrityl tetranitrate, pentaerythritol tetranitrate, sodium nitroprusside, milrinone, inamrinone (formerly amrinone), cilostazol, sildenafil, tadalafil, minoxidil, aliskiren, nitric oxide, sodium nitrite, nitroglycerin, and analogs, derivatives, prodrugs, and pharmaceutically acceptable salts thereof.

In some embodiments, the active agent is an anti-restenosis or restenosis inhibiting agent. Suitable anti-restenosis agents include: (1) antiplatelet agents including: (a) thrombin inhibitors and receptor antagonists, (b) adenosine disphosphate (ADP) receptor antagonists (also known as purinoceptor₁ receptor antagonists), (c) thromboxane inhibitors and receptor antagonists and (d) platelet membrane glycoprotein receptor antagonists; (2) inhibitors of cell adhesion molecules, including (a) selectin inhibitors and (b) integrin inhibitors; (3) anti-chemotactic agents; (4) interleukin receptor antagonists (which also serve as anti-pain/anti-inflammation agents); and (5) intracellular signaling inhibitors including: (a) protein kinase C (PKC) inhibitors and protein tyrosine kinase inhibitors, (b) modulators of intracellular protein tyrosine phosphatases, (c) inhibitors of src homology₂ (SH2) domains, and (d) calcium channel antagonists. Exemplary specific restenosis-inhibiting agents include microtubule stabilizing agents such as rapamycin, mitomycin C, TAXOL®, paclitaxel (i.e., paclitaxel, paxlitaxel analogs, or paclitaxel derivatives, and mixtures thereof). For example, derivatives suitable for use in the stent include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

In some embodiments, the active agent is an anti-coagulation agent. As used herein, the term “anti-coagulation agent” refers to any molecule or composition that promotes blood coagulation or activates the blood coagulation cascade or a portion thereof. Exemplary anti-coagulation agents include, for example, phospholipids such as, e.g., negatively charged phospholipids; lipoproteins such as, e.g., thromboplastin, and the like; proteins such as tissue factor, activated serin proteases such as Factors IIa (thrombin), VII, VIIa, VIII, IX, IXa, Xa, XIa, XII, XIIa, von Willebrand factor (vWF), protein C, snake venoms such as PROTAC® enzyme, Ecarin, Textarin, Noscarin, Batroxobin, Thrombocytin, Russell's viper venom (RVV), and the like; polyvalent cations; calcium ions; tissue factor; silica; kaolin; bentonite; diatomaceous earth; ellagic acid; celite; and any mixtures thereof.

In some embodiments, the agent is a nitric oxide or a prodrug thereof.

Without wishing to be bound by a theory, incorporating an active agent in the bulk material of the stent enables the delivery of active agent in a controlled released manner. Maintaining the active agent in an active form throughout the process of incorporating the agent in the silk fibroin-fibroin matrix enables it to be active upon release from the stent. Controlled release of the active agent permits active agent to be released sustainably over time, with controlled release kinetics. In some instances, the active agent is delivered continuously to the site where treatment is needed, for example, over several weeks. Controlled release over time, for example, over several days or weeks, or longer, permits continuous delivery of the bioactive agent to obtain preferred treatments. The controlled delivery is advantageous because it protects the bioactive agent from degradation in vivo in body fluids and tissue, for example, by proteases.

Controlled release of the active agent from the stent can be designed to occur over time, for example, over 12 hours or 24 hours. The time of release may be selected, for example, to occur over a time period of about 12 hours to 24 hours; about 12 hours to 42 hours; or, e.g., about 12 to 72 hours. In another embodiment, release can occur for example on the order of about 1 day to 15 days. The controlled release time can be selected based on the condition treated. For example, longer times can be more effective for wound healing, whereas shorter delivery times can be more useful for some cardiovascular applications.

Controlled release of the active agent from the stent in vivo can occur, for example, in the amount of about 1 ng to 1 mg/day. In other embodiments, the controlled release can occur in the amount of about 50 ng to 500 ng/day, about 75 ng to 250 ng/day, about 100 ng to 200 ng/day, or about 125 ng to 175 ng/day.

In some embodiments, the active agent is an enzyme that hydrolyzes silk fibroin. Without wishing to be bound by a theory, such enzymes can be used to control the degradation of the stent after implantation into a subject. Controlled degradation of silk fibroin-fibroin based scaffolds with enzymes embedded therein is described in, for example, U.S. Provisional Application No. 61/791,501, filed Mar. 15, 2013, content of which is incorporated herein by reference in its entirety.

In some embodiment, the bulk material of the stent can comprise a cell. Stent with the bulk material comprising a cell can be used for organ repair, organ replacement or regeneration. Cells amenable to be incorporated into the composition include, but are not limited to, stem cells (embryonic stem cells, mesenchymal stem cells, neural stem cells, bone-marrow derived stem cells, hematopoietic stem cells, and induced pluripotent stem cells); pluripotent cells; chrondrocytes progenitor cells; pancreatic progenitor cells; myoblasts; fibroblasts; chondrocytes; keratinocytes; neuronal cells; glial cells; astrocytes; pre-adipocytes; adipocytes; vascular endothelial cells; hair follicular stem cells; endothelial progenitor cells; mesenchymal cells; smooth muscle progenitor cells; osteocytes; parenchymal cells such as hepatocytes; pancreatic cells (including Islet cells); cells of intestinal origin; and combination thereof, either as obtained from donors, from established cell culture lines, or even before or after molecular genetic engineering. Without limitations, the cells useful for incorporation into the composition can come from any source, for example human, rat or mouse. In some embodiments, the cell can from a subject into which the stent is to be implanted.

In some embodiments, the cell is a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

Differentiated cells that have been reprogrammed into stem cells can also be used. For example, human skin cells reprogrammed into embryonic stem cells by the transduction of Oct3/4, Sox2, c-Myc and Klf4 (Junying Yu, et. al., Science, 2007, 318, 1917-1920 and Takahashi K. et. al., Cell, 2007, 131, 1-12).

When using a stent with cells, it can be desirable to add other materials to promote the growth, differentiation or proliferation of the cell. Exemplary materials known to promote cell growth include, but not limited to, cell growth media, such as Dulbecco's Modified Eagle Medium (DMEM), fetal bovine serum (FBS), non-essential amino acids and antibiotics, and growth and morphogenic factors such as fibroblast growth factor (e.g., FGF 1-9), transforming growth factors (TGFs), vascular endothelial growth factor (VEGF), epidermal growth factor (EGF), platelet derived growth factor (PDGF), insulin-like growth factor (IGF-I and IGF-II), bone morphogenetic growth factors (e.g., BMPs 1-7), bone morphogenetic-like proteins (e.g., GFD-5, GFD-7, and GFD-8), transforming growth factors (e.g., TGF-α, TGF-β I-III), nerve growth factors, and related proteins. Growth factors are known in the art, see, e.g., Rosen & Thies, CELLULAR & MOL. BASIS BONE FORMATION & REPAIR (R.G. Landes Co.).

Optionally, the conformation of the silk fibroin in the stent can be altered before, during or after formation of the stent. The induced conformational change alters the crystallinity of the silk fibroin, e.g., Silk fibroin II beta-sheet crystanllinity. Without wishing to be bound by a theory, it is believed that degradation of the bulk material or optional release of an additive (e.g., an active agent) from the bulk material varies with the beta-sheet content of the silk fibroin. The conformational change can be induced by any methods known in the art, including, but not limited to, alcohol immersion (e.g., ethanol, methanol), water annealing, shear stress (e.g., by vortexing), ultrasound (e.g., by sonication), pH reduction (e.g., pH titration and/or exposure to an electric field) and any combinations thereof. For example, the conformational change can be induced by one or more methods, including but not limited to, controlled slow drying (Lu et al., 10 Biomacromolecules 1032 (2009)); water annealing (Jin et al., Water-Stable Silk fibroin Films with Reduced β-Sheet Content, 15 Adv. Funct. Mats. 1241 (2005); Hu et al. Regulation of Silk fibroin Material Structure by Temperature-Controlled Water Vapor Annealing, 12 Biomacromolecules 1686 (2011)); stretching (Demura & Asakura, Immobilization of glucose oxidase with Bombyx mori silk fibroin by only stretching treatment and its application to glucose sensor, 33 Biotech & Bioengin. 598 (1989)); compressing; solvent immersion, including methanol (Hofmann et al., Silk fibroin as an organic polymer for controlled drug delivery, 111 J Control Release. 219 (2006)), ethanol (Miyairi et al., Properties of b-glucosidase immobilized in sericin membrane. 56 J. Fermen. Tech. 303 (1978)), glutaraldehyde (Acharya et al., Performance evaluation of a silk fibroin protein-based matrix for the enzymatic conversion of tyrosine to L-DOPA. 3 Biotechnol J. 226 (2008)), and 1-ethyl-3-(3-dimethyl aminopropyl) carbodiimide (EDC) (Bayraktar et al., Silk fibroin as a novel coating material for controlled release of theophylline. 60 Eur J Pharm Biopharm. 373 (2005)); pH adjustment, e.g., pH titration and/or exposure to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Content of all of the references listed above is incorporated herein by reference in their entirety.

In some embodiments, the conformation of the silk fibroin can be altered by water annealing. Without wishing to be bound by a theory, it is believed that physical temperature-controlled water vapor annealing (TCWVA) provides a simple and effective method to obtain refined control of the molecular structure of silk fibroin biomaterials. The silk fibroin materials can be prepared with control of crystallinity, from a low content, using conditions at 4° C. (a helix (alpha-helix) dominated silk fibroin I structure), to highest content of ˜60% crystallinity at 100° C. (β-sheet (beta-sheet) dominated silk fibroin II structure). This physical approach covers the range of structures previously reported to govern crystallization during the fabrication of silk fibroin materials, yet offers a simpler, green chemistry, approach with tight control of reproducibility. Temperature controlled water vapor annealing is described, for example, in Hu et al., Regulation of Silk fibroin Material Structure By Temperature Controlled Water Vapor Annealing, Biomacromolecules, 2011, 12(5): 1686-1696, content of which is incorporated herein by reference in its entirety.

In some embodiments, alteration in the conformation of the silk fibroin can be induced by immersing in alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is 100%. If the alteration in the conformation is by immersing in a solvent, the silk fibroin composition can be washed, e.g., with solvent/water gradient to remove any of the residual solvent that is used for the immersion. The washing can be repeated one, e.g., one, two, three, four, five, or more times.

In some embodiments, the alteration in the conformation of the silk fibroin can be induced with sheer stress. The sheer stress can be applied, for example, by passing the silk fibroin composition through a needle. Other methods of inducing conformational changes include applying an electric field, applying pressure, or changing the salt concentration.

The treatment time for inducing the conformational change can be any period of time to provide a desired silk fibroin II (beta-sheet crystallinity) content. In some embodiments, the treatment time can range from about 1 hour to about 12 hours, from about 1 hour to about 6 hours, from about 1 hour to about 5 hours, from about 1 hour to about 4 hours, or from about 1 hour to about 3 hours. In some embodiments, the sintering time can range from about 2 hours to about 4 hours or from 2.5 hours to about 3.5 hours.

When inducing the conformational change is by solvent immersion, treatment time can range from minutes to hours. For example, immersion in the solvent can be for a period of at least about 15 minutes, at least about 30 minutes, at least about 1 hour, at least about 2 hours, at least 3 hours, at least about 6 hours, at least about 18 hours, at least about 12 hours, at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days. In some embodiments, immersion in the solvent can be for a period of about 12 hours to about seven days, about 1 day to about 6 days, about 2 to about 5 days, or about 3 to about 4 days. In one embodiment, immersion in the solvent can be for a period of about minutes.

Without limitations, the silk fibroin in the stent can comprise a silk fibroin II beta-sheet crystallinity content of at least about 5%, at least about 10%, at least about 20%, at least about 30%, at least about 40%, at least about 50%, at least about 60%, at least about 70%, at least about 80%, at least about 90%, or at least about 95% but not 100% (i.e., all the silk fibroin is present in a silk fibroin II beta-sheet conformation). In some embodiments, silk fibroin in the stent is present completely in a silk fibroin II beta-sheet conformation, i.e., 100% silk fibroin II beta-sheet crystallinity.

In some embodiments, the stent can be porous, i.e., the bulk material can comprise pores, such as micropores. Without wishing to be bound by a theory, it is believed that pores in the bulk material can allow the stent to function as an endovascular stent graft to prevent rupturing of weakened blood vessel walls. For example, the bulk material of the stent can have a porosity of at least about 10%, at least about 20%, at least about 30%, at least about 40%, at least about 50%, at least about 60%, at least about 70%, at least about 80%, at least about 90%, or higher. One of skill in the art can adjust the porosity accordingly, based on a number of factors such as, but not limited to, desired physical or mechanical properties of the stent, release rates, molecular size and/or diffusion coefficient of the molecule distributed in the bulk material, and/or concentrations, amounts of silk fibroin in the bulk material. As used herein, the term “porosity” is a measure of void spaces in a material and is a fraction of volume of voids over the total volume, as a percentage between 0 and 100% (or between 0 and 1). Determination of porosity is well known to a skilled artisan, e.g., using standardized techniques, such as mercury porosimetry and gas adsorption, e.g., nitrogen adsorption.

The pores can be of any desired pore size. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the pores can have a size distribution ranging from about 50 nm to about 1000 μm, from about 250 nm to about 500 μm, from about 500 nm to about 250 μm, from about 1 μm to about 200 μm, from about 10 μm to about 150 μm, or from about 50 μm to about 100 μm. In some embodiments, the stent can be swellable when hydrated. The sizes of the pores can then change depending on the water content in the stent. In some embodiment, the pores can be filled with a fluid such as water or air.

Methods for forming pores in silk fibroin-based scaffolds are known in the art and include, but are not limited, porogen-leaching methods, freeze-drying methods, and/or gas-forming method. Exemplary methods for forming pores in a silk fibroin-based material are described, for example, in U.S. Pat. App. Pub. Nos.: US 2010/0279112 and US 2010/0279112; U.S. Pat. No. 7,842,780; and WO2004062697, content of all of which is incorporated herein by reference in its entirety.

Though not meant to be bound by a theory, a stent's porosity, structure, and mechanical properties can be controlled via different post-spinning processes such as vapor annealing, heat treatment, alcohol treatment, air-drying, lyophilization and the like. Additionally, any desirable release rates, profiles or kinetics of a molecule encapsulated in the stent can be controlled by varying processing parameters, such as stent thickness, silk fibroin molecular weight, concentration of silk fibroin in the bulk material, beta-sheet conformation structures, silk fibroin II beta-sheet crystallinity, or porosity and pore sizes.

The polymeric sheet for the ratcheting stent design (e.g., stents 100, 200, 300, 400) can be made using any method known in the art for preparing films, e.g., films comprising silk fibroin. As used herein, the term “film” refers to a flat or tubular flexible structure. It is to be noted that the term “film” is used in a generic sense to include a web, film, sheet, laminate, or the like. In some embodiments, the film is a patterned film, e.g., nanopatterned film. Exemplary methods for preparing films comprising silk fibroin are described in, for example, WO 2004/000915 and WO 2005/012606, content of both of which is incorporated herein by reference in its entirety.

The folding stent designs (e.g., stent 500) can be made using any method known in the art for preparing a cylindrical silk fibroin matrix, e.g., silk fibroin tube. For example, tubes can be made using molding, dipping, electrospinning, gel spinning, and the like. Gel spinning is described in Lovett et al. (Biomaterials, 29(35):4650-4657 (2008)) and the construction of gel-spun silk fibroin tubes is described in PCT application no. PCT/US2009/039870, filed Apr. 8, 2009, content of both of which is incorporated herein by reference in their entirety. Construction of silk fibroin tubes using the dip-coating method is described in PCT application no. PCT/US2008/072742, filed Aug. 11, 2008, content of which is incorporated herein by reference in its entirety. Construction of silk fibroin tubes using the film-spinning method is described in PCT application No. PCT/US2013/030206, filed Mar. 11, 2013 and U.S. Provisional application No. 61/613,185, filed Mar. 20, 2012, content of both of which is incorporated herein by reference.

The stent disclosed herein can comprise any desired mechanical stiffness. For example, the stent can comprise an average mechanical stiffness of about 0.01 kN/m² to about 100 kN/m². In some embodiments, the stent can comprise an average mechanical stiffness of from about 0.05 kN/m² to about 75 kN/m², from about 0.1 kN/m² to about 50 kN/m², from about 0.25 kN/m² to about 25 kN/m², from about 0.5 kN/m² to about 10 kN/m², or from about 0.75 kN/m² to about 2 kN/m². In one embodiment, the stent has an average mechanical stiffness of about 1.2 kN/m².

The radial strength of the stent can also be optimized for any desired application. For example, the stent can have an average radial strength of from about 100 mmHg to about 1000 mmHg. In some embodiments, the stent has an average radial strength of from about 75 mmHg to about 750 mmHg, from about 50 mmHg to about 600 mmHg, from about 100 mmHg to about 500 mmHg, from about 150 mmHg to about 450 mmHg, from about 200 mmHg to about 450 mmHg, or from about 250 mmHg to about 350 mmHg. In some embodiments, the stent has an average radial strength of about 300 mmHg.

Compressive toughness is the capacity of a material to resist fracture when subjected to axially directed pushing forces. By definition, the compressive toughness of a material is the ability to absorb mechanical (or kinetic) energy up to the point of failure. Toughness is measured in units of joules per cubic meter (Jm⁻³) and can be measured as the area under a stress-strain curve. In some embodiments, the stent has a compressive toughness of about 1 kJ m⁻³ to about 20 kJm⁻³ or about 1 kJm⁻³ to about 5 kJm⁻³ at 6% strain as measured by the J-integral method.

Compressive strength is the capacity of a material to withstand axially directed pushing forces. By definition, the compressive strength of a material is that value of uniaxial compressive stress reached when the material fails completely. A stress-strain curve is a graphical representation of the relationship between stress derived from measuring the load applied on the sample (measured in MPa) and strain derived from measuring the displacement as a result of compression of the sample. The ultimate compressive strength of the material can depend upon the target site of implantation. In some embodiments, the stent comprise a compressive strength (stress to yield point) of approximately 1 MPa to approximately 10 MPa.

Compressive elastic modulus is the mathematical description of the tendency of a material to be deformed elastically (i.e. non-permanently) when a force is applied to it. The Young's modulus (E) describes tensile elasticity, or the tendency of a material to deform along an axis when opposing forces are applied along that axis; it is defined as the ratio of tensile stress to tensile strain (measured in MPa) and is otherwise known as a measure of stiffness of the material. The elastic modulus of an object is defined as the slope of the stress-strain curve in the elastic deformation region. The stent can comprise a compressive elastic modulus of between approximately 1 MPa and approximately 30 MPa at 5% strain.

In some embodiments, the stent can be bioresorbed after implantation into a subject. As used herein, the term “bioresorbed” or “bioresorption” refers to infiltration of endogenous tissue or cells into an implanted structure, e.g., stent, which permits integration of the implantable structure and tissues, where one or more components of the implanted structure is replaced by new tissue. For example, the stent can degrade as tissue surrounding the target site remodels or regenerates.

In some embodiments, the cylindrical body portion of the stent can be a multilayered cylindrical body portion. If a multilayered stent comprises an additive and/or active agent, different layers of the body can comprises same or different additive or active agents. For example, some layers can comprise a first additive (or active agent) and some other layers can comprise a second additive (or active agent). In some embodiments, the outermost layer comprises no active agent. The number of layers in the multilayered cylindrical body portion of the stent can be any desired number. For example, the multilayered cylindrical body portion of the stent can comprise from 1 to 100, 1 to 75, 1 to 50, 1 25, or 1 to 20 layers.

Without limitations, thickness of each layer can range independently from nanometers to millimeters. For example, thickness of layer can be 1 nm to 1000 nm, 1 nm to 500 nm, 1 nm to 250 nm, 1 nm to 100 nm, 1 nm to 50 nm, 1 nm to 25 nm, 1 nm to 10 nm, 1 μm to 1000 μm, 1 μm to 500 μm, 1 μm to 250 μm, 1 μm to 100 μm, 1 μm to 50 μm, or 1 μm to 25 μm. Further all layers can be of the same thickness, all of different thickness, or some of same and some of different thickness.

The stent designs according to the present invention can also incorporate other features of silk fibroin and silk fibroin based polymers, including the ability to load and deliver therapeutic compounds and up to 100% degradability of the stent material over time within the body.

In accordance with some embodiments of the invention, the bulk material can include a silk fibroin:glycerol blend in a dry weight ratio of 75:25. The bulk material can be fabricated as described below. Other plasticizers, in addition to or instead of glycerol, can be used. Other weight ratios can also be used.

In accordance with some embodiments, the bulk silk fibroin material can be formed from Cocoons of the silk fibroinworm Bombyx mori (supplied by Tajima Shoji Co., Yokohama, Japan). Sodium carbonate, lithium bromide, and Slide-a-Lyzer dialysis cassettes can be purchased from Pierce, Inc. (Rockford, Ill., US). Silk fibroin solutions can be prepared by processing the silk fibroin cocoons. The B. mori silk fibroin cocoons can be boiled in 0.02M aqueous Na₂CO₃ for 30 minutes to extract the sericin component and isolate the silk fibroin protein. The isolated silk fibroin can then be washed three times for 20 minutes in deionized water and allowed to dry for 48 hours at room temperature. The dried silk fibroin can be dissolved in 9.3M LiBr at 60° C. for 4 h, and the resulting 20% (w/v) solution can be dialyzed against water using a Slide-a-Lyzer dialysis cassette (molecular weight cutoff 3500) for two days to remove salts. The resulting concentration of aqueous silk fibroin ranged from 5-7% (w/v), which was calculated by weighing the remaining solid after drying. The aqueous silk fibroin solution can be concentrated by exposing the cassette membrane to ambient air for varying times to produce a 10-20% (w/v) silk fibroin aqueous solution. Deionized water can be blended with the silk fibroin solutions to bring concentrations below 5%. The silk fibroin solutions can be stored at 4° C. until use. Aqueous fibroin solution prepared as described above can be used to cast sheet and tubular films as described above. The films can be fabricated by blending aqueous fibroin with 99% (w/v) glycerol to produce blends of 75:25 (dry weight) silk fibroin:glycerol solution.

In accordance with embodiments of the present invention, the stents can be deployed using standard balloon catheters using familiar surgical procedures, in particular wire guidance and balloon inflation. Prior art natural polymer stents are traditionally incompatible with balloon deployment devices due to the difference in ductility and plastic deformability compared to metal counterparts. The stents according to the invention can be deployed using standard plastic and silicone deployment devices currently clinically used. In accordance with some embodiments of the invention, the different stent designs can make use of a different mechanism when mounting the stent onto the catheter, and subsequently a different mechanism of dilation during deployment. This does not impact the surgeon's standard procedures at the time of deployment.

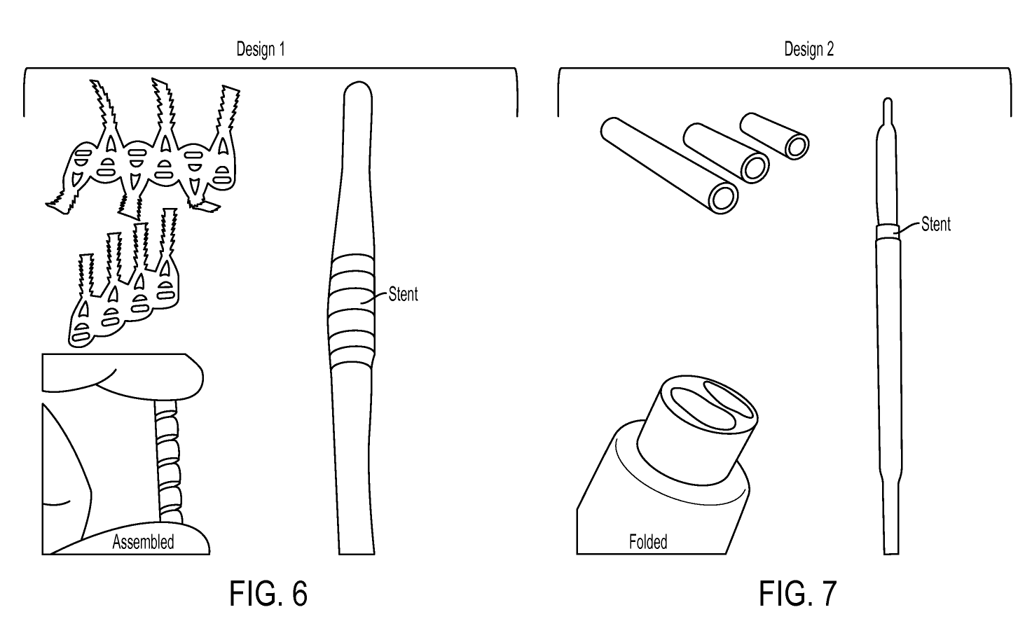

In accordance with some embodiments of the invention, the ratcheting stent can be assembled around a standard radiopaque balloon catheter and tightened to its minimal diameter. The struts or tabs can be wrapped around the stent and catheter then the assembly can be covered with a standard catheter sheath as shown in FIG. 6. The catheter tip can be guided to the target site using standard wire guiding and imaging techniques. Next, the sheath can be retracted and the balloon can be inflated to expand the stent using standard pneumatic pressure regulation. As the stent progressively expands, the one-way ratchet mechanism allows the diameter to increase easily and then resists radial compression after the balloon is removed. The ratcheting stent effectively locks into the desired diameter instantly after deployment and does not require over-dilation or long periods of dilation due to the lack of radial recoil which is associated with clinically available metal stents. Individual stent elements arranged along the axial length of the stent allow for deployment into irregular vessels and can provide a configuration wherein different stent elements are expanded to different diameters.

In accordance with some embodiments of the invention, the folding stent 500 can be mounted around a standard radiopaque balloon catheter using a folded-wrap technique. The folding stent 500 can be wrapped around the catheter balloon then the assembly can be covered with a modified catheter sheath as shown in FIG. 7. The sheath can span the length of the catheter to the balloon where the sheath diameter can be increased slightly to form a seat and casing for the mounted stent. The catheter tip can be guided to the target site using standard wire guiding and imaging techniques. Next, the sheath can be retracted and the balloon can be inflated to expand the stent using standard pneumatic pressure regulation. In accordance with some embodiments of the invention, the stent can respond quickly to the balloon to become fully deployed to its full diameter within 5 seconds (e.g., within 4, 3, or 2 seconds). Using this deployment method avoids exposing the vessel to over-dilation or the long periods of dilation which are associated with clinically available metal stents due to radial recoil.

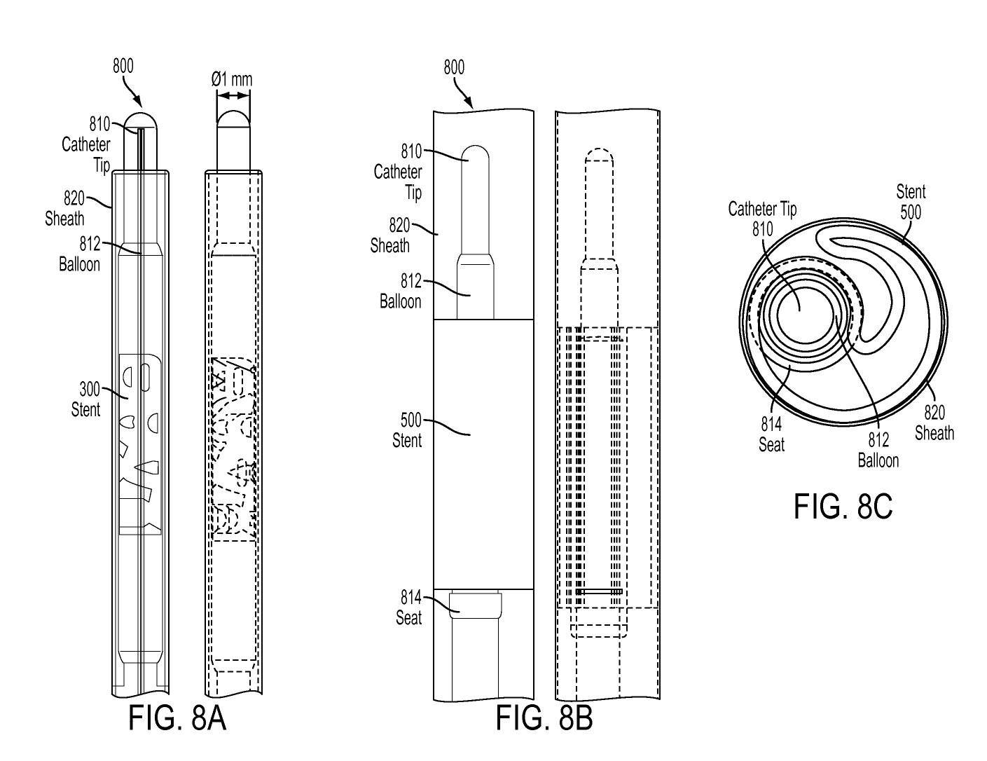

FIG. 8A shows a photograph and a corresponding diagram of a ratcheting stent 300 wrapped tightly around a balloon 812 of a balloon catheter 800. The ratcheting stent 300 can be positioned centrally on the balloon 812 which can be spaced by a predefined distance from the catheter tip 810. A sheath 820 can be positioned over the ratcheting stent 300 and extend adjacent to the catheter tip 810. In operation, the ratcheting stent 300 can be selected from a kit or set of stents of different lengths and maximum diameters. The selected ratcheting stent 300 can be assembled around the balloon 812 at a predefined distance from the catheter tip 810 to allow for imaging techniques to be used to position the catheter tip 810 and the ratcheting stent 300 in the desired location in the vessel. After the position of the ratcheting stent 300 is set, the sheath 820 can be retracted and the balloon 812 can be inflated to expand the ratcheting stent 300 in place to the desired diameter.

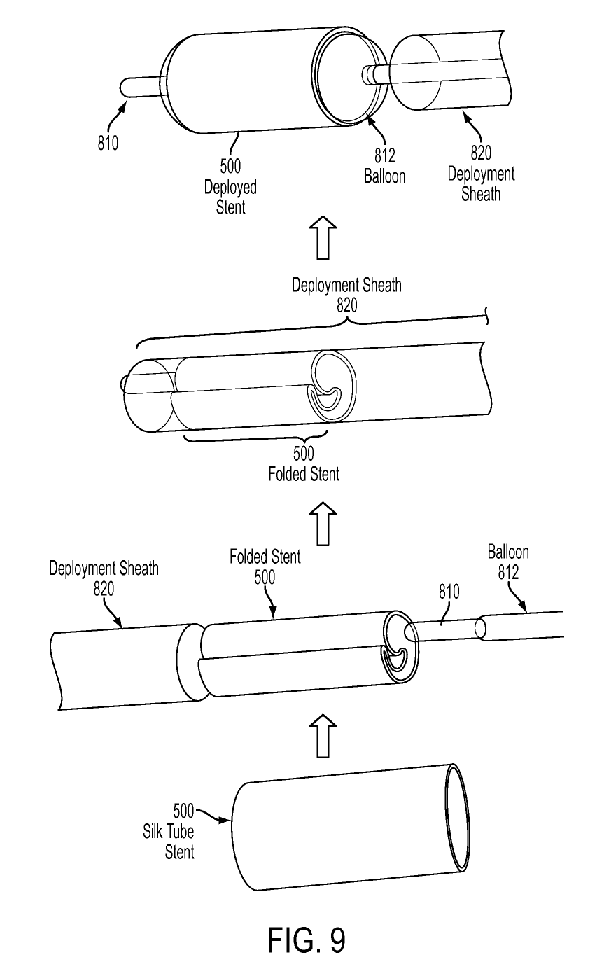

FIGS. 8B and 8C show a photograph and a corresponding diagrams of a folding stent 500 wrapped tightly around a balloon 812 of a balloon catheter 800. The folding stent 500 can be positioned centrally on the balloon 812 which can be spaced by a predefined distance from the catheter tip 810. A sheath 820 can be positioned over the folding stent 500 and extend adjacent to the catheter tip 810. In operation, as shown in FIG. 9, the silk fibroin polymer folding stent 500 can be selected from a kit or set of stents of different lengths and maximum diameters. The selected folding stent 500 can be folded around the balloon 812 (e.g., as shown in FIG. 8C) at a predefined distance from the catheter tip 810 (as shown in FIG. 8B) to allow for imaging techniques to be used to position the catheter 800 and the folding stent 500 in the desired location in the vessel. After the position of the folding stent 500 is set, the sheath 820 can be retracted and the balloon 812 can be inflated to expand the folding stent 500 in place to the desired diameter.

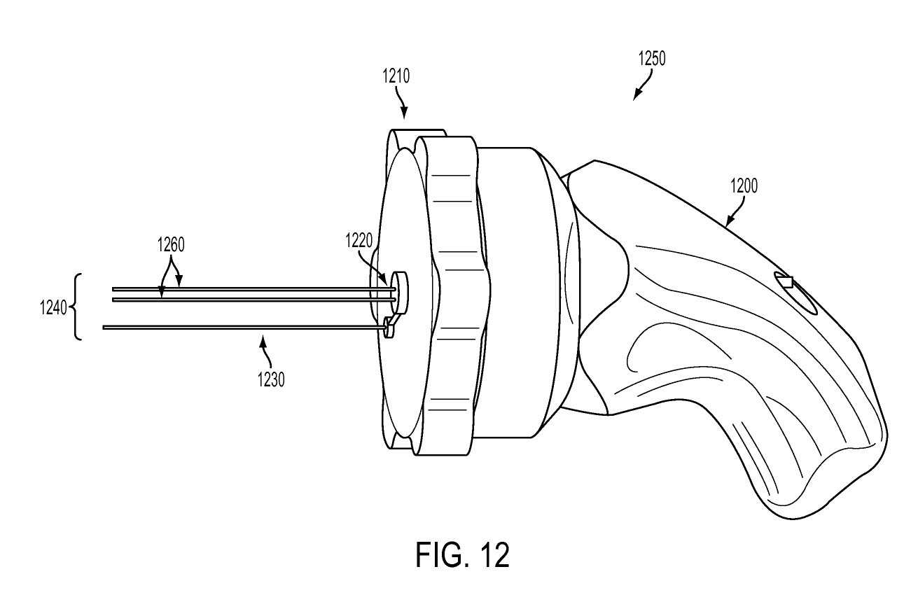

In some embodiments, it may be useful or preferred to employ a stent loader to assist in loading a provided stent, such as a folding stent, onto a balloon catheter and/or into a sheath for deployment. Any application-appropriate stent loader may be used in accordance with the present invention. FIG. 12 shows a profile view of an exemplary stent loader 1250 comprising a handle 1200; a base 1210; a pin holder 1220 which fixedly supports two pins 1260 and which are fixedly attached to the handle 1200 (directly or indirectly); and a spacer pin 1230 which is capable of rotation coincident with that of the base 1210. For ease of reference, the pin holder 1220 and spacer pin 1230 may be collectively referred to as the “pin assembly” 1240. In some embodiments, the spacer pin 1230 is fixedly attached to the base 1210 to facilitate rotation. In some embodiments, the pins 1260 may be attached to a pin holder 1220 that is in turn fixedly attached to the handle 1200. In some embodiments, the handle 1200 and base 1210 can be held together by a pin holder 1220 that screws into the handle 1200, for example, through an opening in the base 1210.

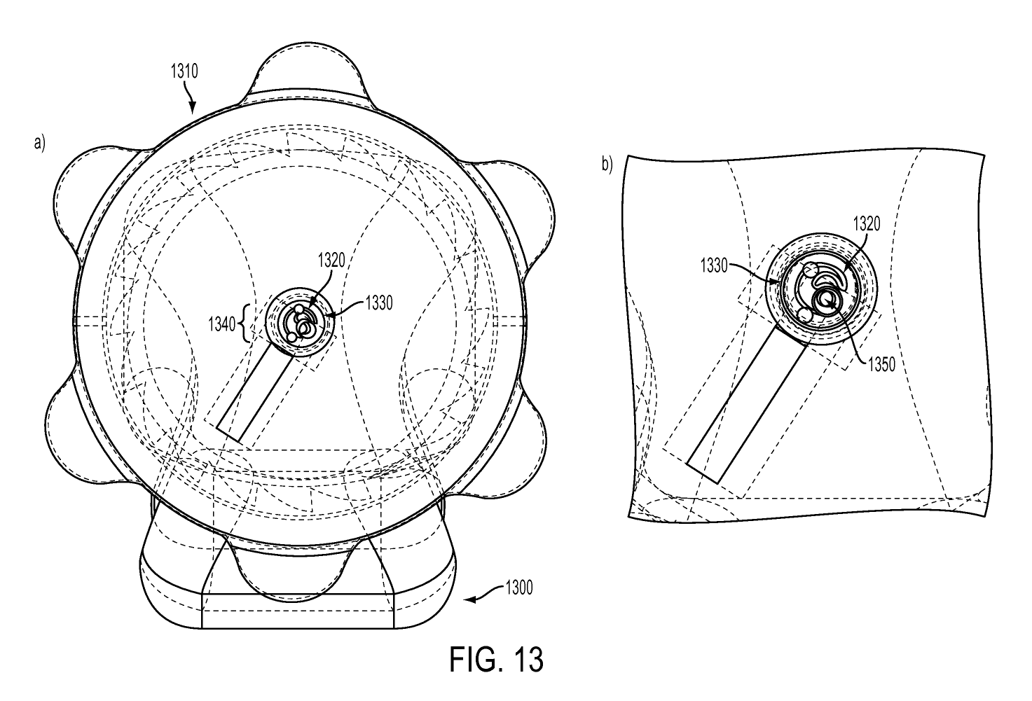

FIG. 13A shows a front view of the exemplary stent loader of FIG. 12, showing, among other things, the handle 1300 and base 1310 as well as the pin assembly 1340. FIG. 13A also shows a front view cross-section of a folded stent 1320 and a sheath 1330. FIG. 13B shows a close up version of the pin assembly 1340, folded stent 1320, and sheath 1330. The handle may have ergonomic finger grips for ease of use, and to allow for one-handed grip.

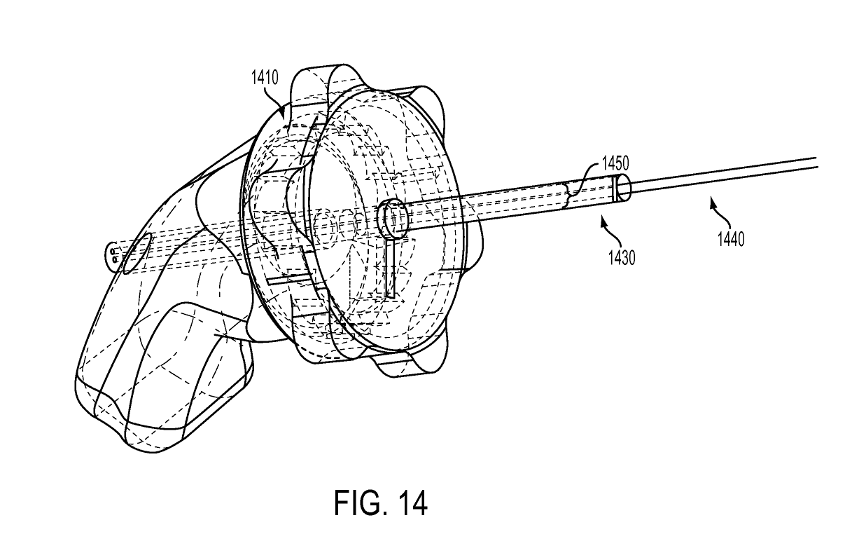

FIG. 14 shows a reverse isometric view of the stent loader of FIG. 12. Showing another view of the relative positioning of the balloon catheter 1440 and sheath 1430 as compared to the pin assembly 1450.

In some embodiments, a stent loader may be used as follows: first, a user inserts the stent 1320, such as a stent 500 as shown in FIG. 5A, about the three pins of the pin assembly 1240/1340/1450 (with the stent axially threaded within and/or around the pins) and then inserts the loaded assembly into a partially uncovered sheath 1330/1430 surrounding a balloon catheter 1440; next, while holding the balloon catheter 1440 in place. the user rotates the base 1210/1310/1410 which operates via a ratcheting mechanism, thereby rotating the spacer pin 1230/1350 that is attached to the base and folding the catheter 1440 into a folded form around the balloon catheter (such as, for example, a folded form 500 as shown in FIG. 5B); finally, once the stent 1320 is fully folded, the user fully inserts the stent 1320 into the sheath 1330/1430 and then removes the loading tool.

According to various embodiments, the spacing between the stationary pins 1260 and the spacer pin 1230 may be altered to accommodate various clinical scenarios, such as deployment of stents of varying sizes. In particular, the distance between each pin in the pin assembly 1240/1340/1450 may range between 0.1 mm and 10 mm. In some embodiments, the distance between each pin of the pin assembly 1240/1340/1450 may be between: 0.5 mm and 8 mm, 1 mm and 8 mm, 1 mm and 7 mm, 1 mm and 6 mm, 1 mm and 5 mm, 2 mm and 5 mm, 2 mm and 4 mm, 2.5 mm and 3.5 mm, inclusive.

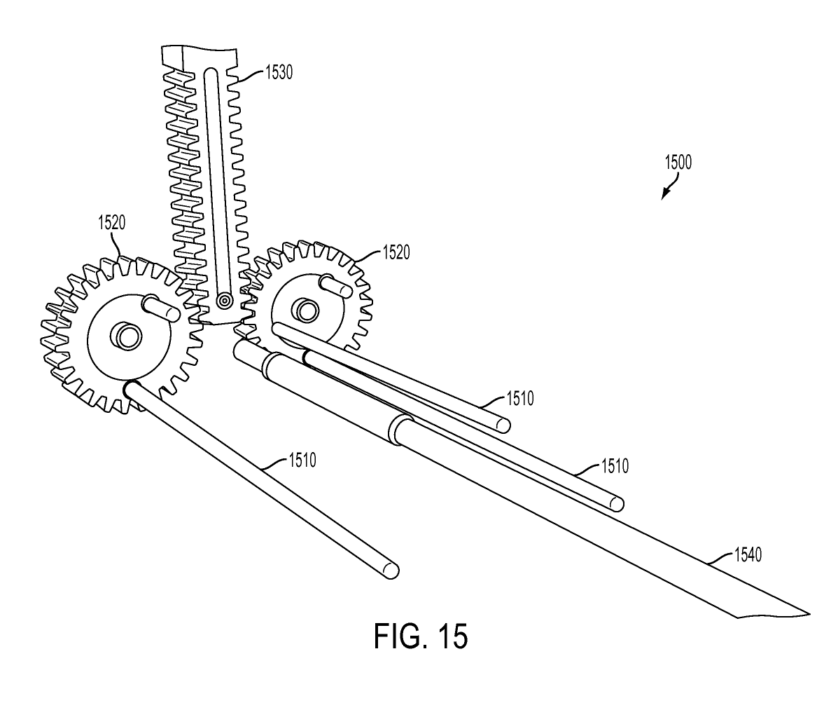

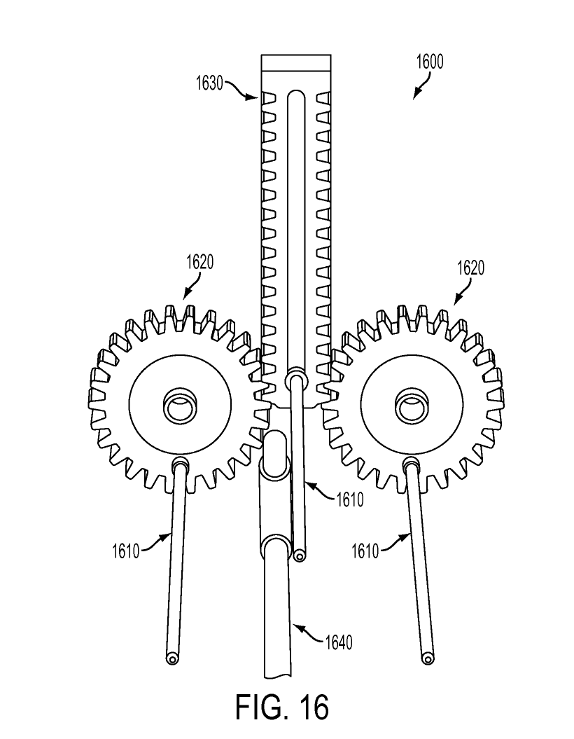

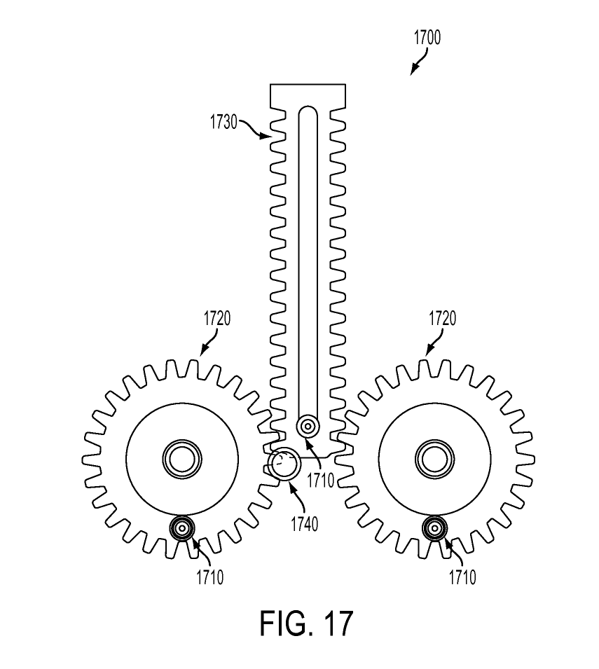

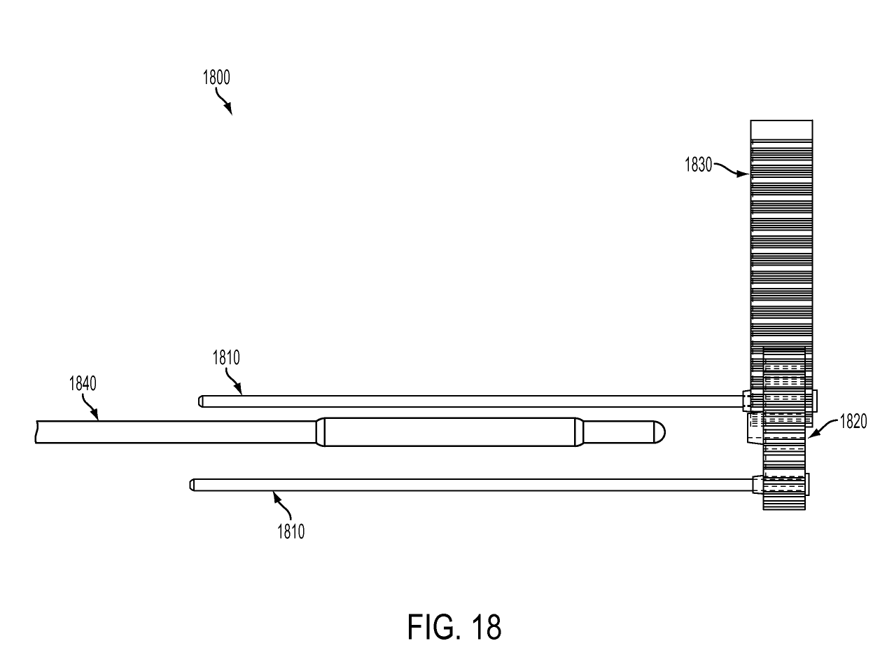

Another exemplary stent loader is shown in FIGS. 15-18. Rather than using a rotational folding mechanism, the exemplary stent loader shown in FIGS. 15-18 employs a pinching mechanism in order to fold provided stents around the deployment catheter. Specifically, the stent loader 1500/1600/1700/1800 includes a gear rack 1530/1630/1730/1830 functionally connected to two gears 1520/1620/1720/1820 and each of the gear rack 1530/1630/1730/1830 and gears 1520/1620/1720/1820 has a prong 1510/1610/1710/1810 fixedly attached thereto. As shown in FIGS. 15-18, the catheter 1540/1640/1740/1840 and stent (not shown) is placed between each of the three prongs 1510/1610/1710/1810 and a user causes downward motion of the gear rack 1530/1630/1730/1830 and associated prong 1510/1610/1710/1810, thus driving coincident motion of the two gears 1520/1620/1720/1820 and the prongs 1510/1610/1710/1810 attached thereto. The simultaneous motion of all three prongs causes the center of the stent (not shown) to be pushed downwards, while the side prongs 1510/1610/1710/1810 attached to each gear 1520/1620/1720/1820 are rotated outward and then over the center of the stent (not shown), thereby folding the stent around the catheter 1540/1640/1740/1840.

According to various embodiments, the components of a stent loader, such as the exemplary stent loaders described above, may comprise or consist of any medically appropriate material including plastics, metals, rubber, or other materials. In some embodiments, a metal may be any sinterable metal such as tungsten, bronze, rhenium, tantalum, osmium, silver, and/or gold or alloys thereof. In some embodiments, a metal may be stainless steel or aluminum. In some embodiments, the components of a stent loader may comprise or consist of any autoclavable material(s). In some embodiments, one or more components of a stent loader may be manufactured using 3D printing.

In accordance with some embodiments of the invention, a one piece solid mesh structure can also be used. This mesh structure design can be deployed using the same balloon deformation mechanism of current metal stenting technology. However, this embodiment may not be able to provide the same amount of radial support as some of the other embodiments described herein.

The ratcheting stent according to some embodiments of the invention provides a one-way mechanical mechanism for discrete expansion, using, for example, a slot and gear or tooth that can function similar to a zip-tie or ratcheting tie wrap. The stent body can contain a slotted portion while the stent struts or strips can include a gear or tooth rack. To avoid potential blood flow obstruction caused by a traditional square ratchet head, the design can be modified to utilize two parallel slots and to use parallel slots with double sided gear racks.

In accordance with some embodiments of the invention, it is desirable to use a bioresorbable material that can be made compliant at the tissue interface and be radially robust as a structural component. Silk fibroin can be used as the base polymer because it possesses these qualities, and can be fabricated aqueously without the need for harsh solvents which would otherwise inhibit drug incorporation later on.

In accordance with some aspects of the invention, the stent body can be fabricated in one piece by excising the body from film sheets using a laser. In some embodiments, the polymer sheets can suffer from significant burn zones near the cut edge which blunts and recedes the edge from the intended cut, and rounds reciprocated cuts. To compensate for and avoid theses defects, the minimum feature size in those burn zones can be enlarged in order to maintain the functionality provided by the ratchet mechanism. Additionally, stent functionality and uniformity of mechanical strength can be improved by increasing the number of tabs and slots, and distributing them symmetrically within the device.

In accordance with some embodiments of the invention, glycerol can be added to the silk fibroin film blend to improve solubility and stent surface compliance, flexibility and resilience. The addition of glycerol can also improve the fabrication process by reducing the defects in the burn zones and sharpening the features of the device.

Uniaxial compressive resistance of initial stent revisions can be highly dependent on the angle relative to the ratchet slot. Further revisions of the stent design can incorporate modifications to the position of the slots and tabs, for example, distributing them symmetrically within the device such that, when assembled, the stent appeared to have radial symmetry, which can make compressive resistance more uniform.

Stent deployment was initially met with difficulty. The ratchet slot design enabled one-way expansion and successfully prevented reverse sliding, but in many balloon dilation trials within silicone tubing resulted in tearing of the stent material rather than smooth expansion. Several revisions were designed to facilitate sliding of the struts without compromising the one-way function. The geometry of the teeth can be changed such that they bent easier in one direction. Rounded tips and additional filleting can also be added to the teeth.

In accordance with some embodiments of the invention, the ratcheting stent design where maximal increase in stent diameter can be desirable, such as when the stent must first traverse through vessels narrower than the target stent vessel. In contrast, the folding stent design can be used where it is desirable to reduce or eliminate the risk of vessel erosion and/or restenosis. However, a slightly different deployment method can be used. The folding stents can be mounted around the balloon catheter and then folded and wrapped around it. However, the folding stent will not remain fixed in this position and so a sheath can be used to keep the folding stent folded and mounted in position around the catheter.

The resistance of the stent to remain folded exerts a force on the inside surface of the sheath which produces enough friction to cause the stent to move away from the target site during retraction of the sheath. A seat at the base of the balloon and attached to the catheter can be used to limit sliding of the stent during sheath retraction.

The tubular silk fibroin material is very strong and the addition of glycerol to the film blend can improve compliance, flexibility and resilience. The formulation of the silk fibroin:glycerol blend can be used to impart plasticizing properties which can avoid creasing of the stent at the points of folding.

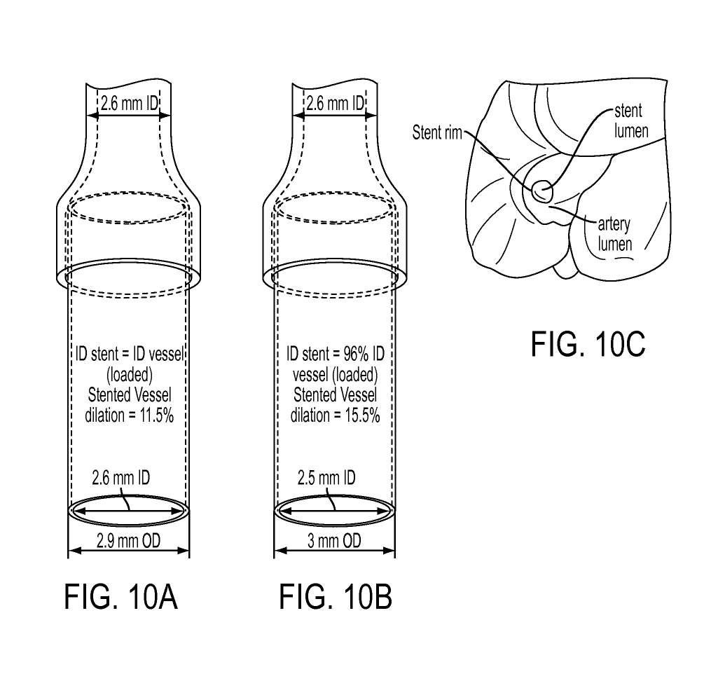

The folding stent design 500 does not expand beyond the intended diameter and there can be select to properly fit to each vessel. In accordance with some embodiments of the invention, a kit or sized assortment having a range of diameters of folding stent 500 be provided to prepare for surgery. The targeted vessel diameters can be clinically evaluated prior to implantation. With this information, stents can be fitted appropriately. As shown in FIG. 10A, malapposition flow studies have found that the folding stents can perform optimally when the stent internal diameter (I.D.) is equal to the loaded I.D. of the blood vessel, and the blood vessel wall is displaced an additional 11.5% by the stent wall. For example, if the vessel I.D. is 2.6 mm, then an appropriately sized folding stent can have an I.D. of approximately 2.6 mm but an outer diameter (O.D.) of 2.9 mm. However, mechanical evaluation has shown stent walls to produce superior strength at a thickness in the range of 250 to 300 μm. According to some embodiments of the invention, optimal stent fitting in the blood vessel from the previous example exhibiting a loaded I.D. of 2.6 mm, would now call for a stent with an I.D. of 2.5 mm but an outer diameter (O.D.) of 3 mm, producing an additional 15.5% dilation rather than 11.5%.

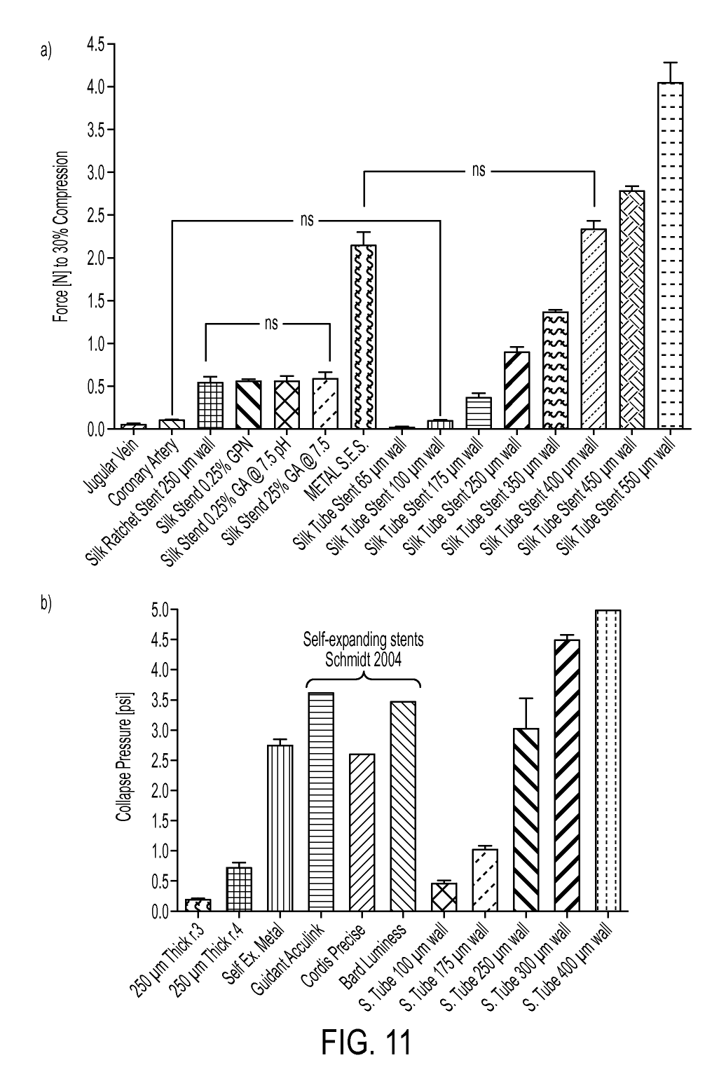

FIG. 11 shows the strength of various embodiments of the present invention and devices and materials in the prior art. Tensile properties were evaluated using a uniaxial mechanical tester (model 3366, Instron Inc., Norwood, Mass., US), with a length between clamps of 35±3 mm. Samples were excised using 12 mm wide rectangular shape. Three samples per group were tested in both simulated physiological conditions in PBS at 37° C., and in ambient air at 22° C. Samples were pre-cycled for three 30 second intervals at a rate of 1 mm/minute, and then pulled at a rate of 1 mm/minute until failure. Uniaxial compression testing was performed with a uniaxial mechanical tester (model 3366, Instron Inc., Norwood, Mass., US). The implants were positioned between two opposing parallel plates. Compressive force was measured during compression until the implant diameter was reduced to 70%. Radial strength was assessed using a pressure chamber. Implants were fitted in latex sleeves and external pressure was increased until collapse. Collapse pressure was measured in pounds per inch.

In accordance with some embodiments of the invention, the polymer stent can be formed of a material that has an average mechanical stiffness in the range from 0.5 kN/m to 3.0 kN/m, in the range from 1.0 kN/m to 3.0 kN/m, in the range from 1.0 kN/m to 2.5 kN/m, in the range from 1.0 kN/m to 2.0 kN/m, in the range from 1.0 kN/m to 1.5 kN/m, and other stiffness ranges can be used. In one embodiment of the invention, the stent can be include a cylindrical body having an average mechanical stiffness of approximately 1.2 kN/m. In accordance with some embodiments of the invention, the polymer stent can be formed of a material that has an average radial strength in the range from 100 mmHg to 500 mmHG, in the range from 100 mmHg to 400 mmHg, in the range from 200 mmHg to 400 mmHg, in the range from 200 mmHg to 300 mmHg, in the range from 250 mmHg to 350 mmHg, and other stiffness ranges can be used. In one embodiment of the invention, the stent can be include a cylindrical body having an average radial strength of approximately 300 mmHg.

Stent malapposition was evaluated using a peristaltic pump. Porcine carotid arteries were excised and mounted inline of a pump-driven aqueous flow loop using barb fittings, and secured circumferentially with silk fibroin sutures. Flow was increased by 100 mL·min-1 each hour until reaching 1600 mL·min-1, which is 7 times greater than physiological flow and velocity. No stent malapposition was detected. Results were supported by repeating the study with only the proximal vessel end mounted to the flow loop, and the other end was left unmounted. Pump was restarted, allowing the stented vessel to discharge freely into the reservoir. No stent malapposition was detected.

References cited in the present disclosure are hereby incorporated by reference in their entirety. Other embodiments are within the scope and spirit of the invention. Features implementing functions may also be physically located at various positions, including being distributed such that portions of functions are implemented at different physical locations.

Further, while the description above refers to the invention, the description may include more than one invention.

What is claimed is:

1. An intraluminal stent implantable in a body lumen comprising:
a cylindrical body portion having an axial length extending along a longitudinal axis, said cylindrical body portion formed of a sheet wherein the cylindrical body portion is formed of a blend comprising silk fibroin and a plasticizer.

2. The intraluminal stent of claim 1, including
a first longitudinal edge and
a second longitudinal edge,
an elongated slot in said sheet along the first longitudinal edge, said elongated slot receiving said second longitudinal edge allowing said cylindrical body portion to be selectively expanded in the body lumen.

3. The intraluminal stent according to claim 2, further comprising a strip connecting the second longitudinal edge to the first longitudinal edge, wherein the strip includes one or more teeth adapted for interlocking the strip in the elongated slot.

4. The intraluminal stent according to claim 3, wherein at least one of the teeth is flexible.

5. The intraluminal stent according to claim 2, further comprising a second slot in the sheet along the first longitudinal edge and a second strip connecting the second longitudinal edge to the first longitudinal edge, wherein the second strip includes one or more teeth adapted for interlocking the strip in the second elongated slot.

6. The intraluminal stent according to claim 1, wherein cylindrical body portion is formed of a blend comprising silk fibroin and a plasticizer in a ratio of 10:1 to about 1:1 by dry weight or a ratio of 3:1 by dry weight.

7. The intraluminal stent according to claim 1 including
at least one tab extending from a first longitudinal edge and
at least one slot extending from the second longitudinal edge to the first longitudinal edge, the slot receiving the at least one tab and allowing the cylindrical body to be selectively expanded in the body lumen; and
wherein the cylindrical body portion is formed of a blend of silk fibroin and a plasticizer.

8. The intraluminal stent according to claim 7, further comprising a second tab extending from the first longitudinal edge and a second slot extending from the second longitudinal edge to the first longitudinal edge, the second slot receiving the second tab and allowing the cylindrical body to be selectively expanded in the body lumen.

9. The intraluminal stent according to claim 7, wherein the at least one slot includes one or more teeth adapted for interlocking the tab in the at least one slot.

10. The intraluminal stent according to claim 9, wherein at least one of the teeth is flexible.

11. The intraluminal stent according to claim 7, wherein cylindrical body portion is formed of a blend of 75% silk fibroin by dry weight and 25% plasticizer by dry weight.

12. An intraluminal stent implantable in a body lumen comprising:
a cylindrical body portion having an axial length extending along a longitudinal axis, said cylindrical body portion formed of a blend of silk fibroin and a plasticizer;
the cylindrical body capable for existing in a folded state having a first diameter and an unfolded state having a second diameter wherein the second diameter is greater than the first diameter.

13. The intraluminal stent according to claim 12, wherein cylindrical body portion is formed of a blend of 75% silk fibroin by dry weight and 25% plasticizer by dry weight.

14. An intraluminal stent implantable in a body lumen comprising a cylindrical body portion having an axial length extending along a longitudinal axis, said cylindrical body portion formed of a blend comprising silk fibroin and a plasticizer; the bend comprising silk fibroin and plasticizer being prepared in a process that includes:
casting the blend of silk fibroin and plasticizer to form a film.

15. The intraluminal stent according to claim 14, wherein the blend of silk fibroin and plasticizer is cast in a sheet in a flat mold.

16. The intraluminal stent according to claim 15 wherein the cast sheet has a thickness in the range of 150 to 300 micrometers.

17. The intraluminal stent according to claim 14, wherein the blend of silk fibroin and plasticizer is cast on a rod to form a tubular film.

18. The intraluminal stent according to claim 17, wherein the cast tubular film has an inside diameter in the range from 650 micrometers to 4500 micrometers.

19. The intraluminal stent according to claim 17, wherein the cast tubular film has a thickness in the range of 150 to 300 micrometers.

20. The intraluminal stent according to any of claims 1-19, wherein the blend comprising silk fibroin and plasticizer further comprises an additive.

21. The intraluminal stent according to claim 20, wherein the additive is selected from the group consisting of anti-proliferative agents, biopolymers, nanoparticles (e.g., gold nanoparticles), proteins, peptides, nucleic acids (e.g., DNA, RNA, siRNA, modRNA), nucleic acid analogs, nucleotides, oligonucleotides, peptide nucleic acids (PNA), aptamers, antibodies or fragments or portions thereof (e.g., paratopes or complementarity-determining regions), antigens or epitopes, hormones, hormone antagonists, growth factors or recombinant growth factors and fragments and variants thereof, cell attachment mediators (such as RGD), cytokines, enzymes, small molecules, antibiotics or antimicrobial compounds, toxins, therapeutic agents and prodrugs, small molecules and any combinations thereof.

22. The intraluminal stent according to any of claims 1-21, wherein the cylindrical body portion is a multilayered cylindrical body portion.

23. The intraluminal stent according to claim 22, wherein a first layer and a second layer of the cylindrical body portion each comprise at least one additive.

24. The intraluminal stent according to any of claims 19-23, wherein degradation of the blend material or optional release of the additive from the blend material varies with the beta sheet content of silk fibroin in the blend material.

25. The intraluminal stent according to any of claims 1-24, wherein the cylindrical body portion has an average mechanical stiffness of 1.2 kN/m.

26. The intraluminal stent according to any of claims 1-25, wherein the cylindrical body portion has an average radial strength of 300 mmHg.

27. The intraluminal stent according to any of claims 1-26, wherein the blend material comprises micropores.

28. The intraluminal stent according to claim 27, wherein the stent functions as an endovascular stent graft to prevent the rupturing of weakened blood vessel walls.

29. The intraluminal stent according to any of claims 1-28, wherein the blend comprising silk fibroin and plasticizer further comprises an active agent.

30. The intraluminal stent according to claim 29, wherein the active agent is a therapeutic agent.

31. The intraluminal stent according to any of claims 1-30, wherein the blend comprising silk fibroin and plasticizer further comprises an anti-restenosis agent.

32. The intraluminal stent according to any of claims 1-31, wherein the blend comprising silk fibroin and plasticizer further comprises a cell.

33. A method comprising:
implanting a first intraluminal stent of any of claims 1-32 at a target site in a subject.

34. The method of claim 33, wherein the target site is a tissue.

35. The method of claim 34, further comprising growing cells in or on the stent.

36. The method of claim 33, wherein the target site is a blood vessel.

37. The method of any of claims 33-36, wherein the silk fibroin-based blend material of the stent swells upon implantation at the target site, thereby reducing stent malapposition post-implantation.

38. The method of any of claims 33-37, wherein the stent comprises a therapeutic agent.

39. The method of claim 38, wherein the therapeutic agent is stabilized, upon implantation, for at least 3 months or longer.

40. The method of any of claims 33-39, wherein the stent degrades as a tissue surrounding the target site remodels or regenerates.

41. The method of any of claims 28-37, further comprising implanting a second intraluminal stent of any of claims 1-32 at the same target site upon degradation of the first intraluminal stent.

42. The intraluminal stent according to any of claims 1-32, wherein the plasticizer is selected from the group consisting of glycerol; ethylene glycol; propylene glycol; glyceryl monostearate; 1,2-butylene glycol; 2,3-butylene glycol; styrene glycol; polyethylene glycols such as diethylene glycol, triethylene glycol, tetraethylene glycol; polyethylene glycols; polyethylene oxides; monopropylene glycol monoisopropyl ether; propylene glycol monoethyl ether; ethylene glycol monoethyl ether; diethylene glycol monoethyl ether; sorbitol lactate; ethyl lactate; butyl lactate; ethyl glycolate; allyl glycolate; monoethanolamine; diethanolamine; triethanolamine; monisopropanolamine; triethylenetetramine; 2-amino-2-methyl-1,3-propanediol; and any combinations thereof.

43. A cell scaffold comprising an intraluminal stent of any of claims 1-32 or 42, wherein the plasticizer is wherein the plasticizer is glycerol and the blend comprising silk fibroin and plasticizer further comprises a cell.
